SEQUENCE LISTING (3 sequences)

SEQ ID NO: 1 - DNA, 500 bp, Pseudomonas aeruginosa
ctgcagggctacgctgagggagttgtagaggaggccgatgacaacaccctgcgcatccggctagcccatggccttcgagtcaaagaagcgtctcccgccgccgatgacgtggccgacgttccgcgtacgccgggggttaagaagccttcgatacgtctgctcgggctgctccagctgctgtggttagaggctggactggccaactggtacccactgatggaaggcaagcgaacgcccgcgaatgttgcctattgggtatctgaagccgcaaagcgtatccgggccagccgaatgacggtgttcgacgtcttgctgatgtcggccaagaagaactcccgcatggcgaagcgcaatgctgaggtcgtggagctggcgcaggagcattcgcgcaggctcattgcggtgtcgccgctggcgtccttcaactcggaacgacgcaatctcttgacgctttctgtctcgggacccttcgggatgccgacgatggacattcgagcgga

SEQ ID NO: 2 - DNA, 763 bp, Pseudomonas aeruginosa
tgcaggatctgggtcaggttgtcgatcaggttctgggtcagcgaattcagagctctcattcgtcctggcactccactgccggttggtctgggaaattaattgggaagggcggcaggccgcgcgccttgtcgagatccgctgcgacctgcaaggccgcctcgagctcgtcgcagccggtggcttgcatgatgttgtagcgctggttcttttcttcgttttcggtcatggccagggccaggtagagactcgggggaaccacacggaagaggtactccttgcccttggccaggagcacgccctcggtgaatttgccgctttccttgcgggccgagagcatcatcgacttctgcgccggcgacagctcgcggaacctggagatcttctctacctcgtctgggggcatgttcaggcacaaccaccactcgatcatgttcagcatcggcgccccggaggctgggatgtcgtcgatgttctgggtggcgagccagaaccaggcgcccagtttccgccacatcttggtgatcttcatggcgtagggcagcagcagcgggtgcttggtgatgatgtgtccctcatcggtgatcttgacgatgggccggcccttgaattggtcgcgttcggcgatgttgtttacggtgttcagcagcgagatgtaggcgatcccgagctgggcggcgtagccttcgcgcgcgtaagtcgcgaaatccaccacggtgaggtcggcttcaggccagggcgtgcctttgcgattgaacatctcgcc

SEQ ID NO: 3 - DNA, 2910 bp, Pseudomonas aeruginosa
atgcgttggaaactcccctggccgaagctggccgcaccgaagctggccgcgtccggcgctggcgatgacgagcagccggacggctggcagcgccacgtcgaggcactgcaccaggccggcatccccgaacccggcacggcggtgcaaggtcacaggccggcgaccatggcagacgagcagacgctgtatgaggtcgcgccgtcgttcgtggaactactgccctgggtggagttcctgccgcagtcgaaggccatgttgctggaggacgggcaatcggtggcggccttttacgagctggtgccgctgggcaccgaaggccgggaacccggctggctcgcgcaggcccgcgatgccttggagaacgcgctgcaggacagtttcgatgaactggacgaaactccctgggtgttgcagctctacgcccaggacgagcccagttttgaccagtacatgcagaccctgcgcgactatgtgcagccgcgcgcccgtgatacagcgttcagcgagttctacctgcgcttcttcggccaccacctgcgcgcggtggccaagccgggcggcctgttcgaggacactgtggtcacacggctgcgctggcgcggccagacccggcgcgtgcgcatggtggtctaccgccgcgtgaccgggcaagggcaaaacagccgtcgcgggcagacgcccgagcagatgctcaatatcgtctgcgaccgcctgtgcggcggactggcgaacgccggcattcaggcccggcgcatggtcgcggcagatgttcatgactggttgctgcgctggttcaacccgcgccccacgttgctcgggcctggggccgaagaccgggagcgcttctatacattggcgcgctatcccgacagtgcggaggaaggcgaggacggcgagatcgagctggcgagcggacgggatttcagccaacggctgttcttcggccagccgcgttccgacgtggcgcacggcacctggcatttcgacggcatgccgcaccgcgtgctgatcaccgaccgcctgcgcatgccgccaggcacgggacacctgaccggcgagacgcgcaagggcgacgccatcaacacgttgttcgaccagatgcccgaggacaccatcctttgcctgacgctggtcgccacgccgcaggatgttctcgaagcggatctcaatcatctggcgaagaaggccgtgggcgaaaccctggcatccgagcagacgctcaaggacgtgcatgaagcccgctccctgatcggcagcgcgcacaagctctatcgtggcacgctggcgttctacctgcgcgggcgcgacgaggcggaactggatcggcgcggcctcgacctggcgaacgtcatgctcaacgccggtttgcagccggttcgcgaagacgacgaggtggcaccgctgaacagctacttgcgctggctgccgtgctgctacaaccccggccaggatcggcgcaagtggtacacccaactgatgttcgcccagcacgcggcgaacctctcgcccgtgtggggccgcgcccaaggtacggggcaccccggcatcacgatgttcaaccgcggcggcgggccgatcaccttcgacccgctcaaccgcctggatcggcagatgaatgcccacctgtttctgttcggcccgacaggttcaggcaagtcggcgaccctcaacaacctcttgaatcaggtcacggcgatctaccgtccccgcctgttcattgtcgaggccggcaacagcttcggcttgttcagcgactttgcccggcgcctgggcctgaccgtgaatcgggtcaagctcgctccgggctcgggcgttaccctggcgccgttcgcggatgcgcgccggctgatcgagacgcccagcgacgtgcagacgctcgacgccgatgcgctggatgaagacctgcctcccgatgctgtggccatggaggcagatgagcagcgcgacgtactgggtgaactggagatcaccgcgaggctgatgatcaccggcggcgaagataaagaagaagcccggatgacgcgagccgaccgctcactgatccgtcagtgcatcctcgatgccgccgagcactgccacagcaaggatggcgagaagcgaaccgttctcacgcgcgatgtgcgcaacgcgctgcgcacgcgcagccaggacccgacgctgccggaaatgcggcgtgtgcgactgctggagatggccgacgccatggacatgttctgccaaggcacggacggcgaaatgttcgaccgcgacggttcgccgtggcccgaagccgacatcaccctggtggatctggcgacctatgcccgcgaaggctacaacgcgcagctctctattgcctacatcagcctgatcagcacggtgaacaacattgccgagcgcgatcagtacctgggccgcccgatcatcaacgtcaccgacgaaggacacatcatcaccaagaacccgctgctcgcaccctacgtggtgaagatcaccaagatgtggcgcaagctgggggcctggttctggctcgccacacaaaacatcgacgacttgccgcgcgctgcagagcccatgctcaacatgatcgagtggtggatctgcctgtcgatgccgcccgatgaggtggagaagatcgcgcggttccgcgaactctcgcctgcgcagaaggcgctgatgctttccgcgcgcaaggaagccgggaagttcaccgagggcgtcatcctctccaagagcctggaagtgctgtttcgggccgtgccaccgagcctctatctcgcgctcgcgcagacggaacccgaggagaaggccgagcgttaccagctcatgcagcaacacggcatcagcgaactcgatgcggccttcaaagtggccgagagaatcgaccaggcgcgcggcatcaagtcgccagccgtgggcctgccgcaatag

(19) United States
(12) Patent Application Publication
(10) Pub. No.: US 2006/0008477 A1
(43) Pub. Date: Jan. 12, 2006

(54) Phenotype-determining virulence genes of pseudomonas aeruginosa for colonization and persistence in human beings, animals and plants, uses of said gene and associated proteins

(76) Inventors: Burkhard Tümmler, Hannover (DE); Lutz Wiehlmann, Hannover (DE); Claudia Kiewitz, Inzlingen (DE); Karen Larbig, Hannover (DE); Margit Ritzka, Hannover (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/489,427

(22) PCT Filed: Sep. 12, 2002
(86) PCT No.: PCT/DE02/03393

§ 371 Date: Nov. 19, 2004

(30) Foreign Application Priority Data
Sep. 13, 2001 (DE) 101 45 042.7

(51) Int. Cl. A61K 39/108; C07H 21/04; C12N 1/20; C07K 14/21

(52) U.S. Cl. 424/260.1; 435/252.34; 530/350; 536/23.7

(57) ABSTRACT

Genes of the microorganism Pseudomonas aeruginosa are described which, when switched off, impair the virulence of said microorganism and even render it completely harmless. The genes all of which are known to be essential for the survivability of said microorganism in humans and animals are proposed as targets for the development of medicaments and vaccines for blocking or switching off said genes or the corresponding gene products in vivo and also for the development of diagnostic agents.

The invention relates to newly identified sequences of the microorganism Pseudomonas aeruginosa, to uses of these novel and known sequences of said microorganism and also to corresponding antibodies and vaccines.

The genus Pseudomonas comprises rod-like, polarly flagellated, Gram-negative bacteria. The species Pseudomonas aeruginosa (aerugo, latin for verdigris) are bacteria of 1.5-3 μm in length and 0.5-0.8 μm in diameter which are classified to the group of γ proteobacteria, said species being a relatively common, well-studied and completely sequenced microorganism. The taxonomic classification is carried out on the basis of the 16S rRNA sequences. P. aeruginosa is distinguished by the production of various dyes which are the reason for the name given to the bacterium.

P. aeruginosa has only very low demands on its habitat and, by selectively regulating its gene expression, is capable of adapting to a broad spectrum of different environmental conditions. It has even been detected in almost pure water (>71 kΩ) and in some disinfectants. This high adaptability makes P. aeruginosa an almost ubiquitous and partially dangerous germ which is very sensitive only to drying out. Due to selective regulation of gene expression, the phenotype of said bacteria can vary between mucoid and a nonmucoid variant, depending on the requirements of the habitat.

The nonmucous form of the bacterium is flagellated and provided with a varied surface, thus enabling it to access new habitats. Mucous P. aeruginosa are nonflagellated and differ from the mobile variant in many phenotypic features. The most striking difference here is strong alginate production. This glycocalyx presumably mediates stable adhesion of the bacteria to surfaces and thus ensures the colony can hold out against water currents or ciliary movements of a colonized epithelium. Furthermore, phagocytosis is made more difficult and uptake of nutrients is facilitated (BOTZENHARDT & DÖRING 1993; COSTERTON ET AL. 1987).

Genetic studies have shown that in P. aeruginosa PAO the genes belonging to a metabolic chain are usually not located in polycistronic gene cassette, are not regulated by a shared promoter and are not transcribed as a single mRNA, as is the case, for example, in Escherichia coli, but are encoded at various sites of the genome, despite a common regulation. Studying the PAO genome reveals a two-way split already known from P. putida: essential and anabolic genes are usually located close to the origin of replication, with catabolic and nonessential ones mostly located in the other half. This division can presumably be attributed to new genetic elements, for example plasmids, being integrated into a previously smaller genome. The complete genomic sequence of P. aeruginosa PAO was published in 2000 and is now accessible for further in silico studies (www.pseudomonas.com).

There exists high genetic similarity of strains from very different habitats, which can be explained either by an evolutionary very recent radiation or by a genome organization conserved in principle independently of the habitat. When studying isolates from P. aeruginosa-infected CF patients, it was often possible to detect, in addition to acquisition from the environment, a nosocomial infection within a hospital or infection of likewise affected relatives.

However, P. aeruginosa attacks humans only when their immune system has been weakened generally or locally. The most affected patient groups are premature babies and immunosuppressed patients and also individuals suffering from diseases such as cystic fibrosis (CF), severe burns or malignomas. Owing to its adaptability, its high resistance to antibiotics and its low nutrient requirements, P. aeruginosa can also be found in hospitals, where it is one of the main pathogens of nosocomial diseases, with a proportion of 10.1% according to data of the National Nosocomial Infections Surveillance System (USA) (BRAVENY, KRUMP-SCHMIDT 1985; POLLAK 1985; HORAN 1986).

While colonizing tissue and invading epithelial cells, P. aeruginosa produces a multiplicity of virulence determinants. These include, inter alia, two toxic proteins, exotoxin A and exoenzyme S which, after uptake into eukaryotic cells, cause the death of the latter. Various proteases (elastase, alkaline protease, LasA fragment) destroy locally the tissue structure in the host and hydrolyze immunoglobulins, components of the complement system and receptors of the immunocompetent cells. In addition, P. aeruginosa also secretes heat-stable cytotoxins with detergent-like properties, the “rhamnolipids”, which, owing to their chemical structure, neither can be degraded by proteases of the host nor induce an immune response. Expression of the P. aeruginosa pathogenicity factors is regulated to a large extent in a growth-dependent manner via quorum sensing (DEKIEVIT & IGLEWSKI 2000; STOREY ET AL. 1998).

In contrast to the above-described acute infection, chronic Pseudomonas infection in CF patients is restricted to the lung where it is a decisive factor for the course of the disease. Alginate and mucin can ultimately close whole sections of the lung, resulting in an irreversible conversion of lung tissue in the atelectases thus produced. As a result, the area available for respiration decreases more and more and patients die of pulmonary insufficiency (PIER 1985; HØIBY 1986).

There is therefore a great need for obtaining therapeutics and vaccines for pathogenic Pseudomonas aeruginosa strains and also diagnostic agents which can be used to determine the pathogenicity in detail.

The goal of an immunization must be an immune response which the bacteria cannot escape from by altering their surface proteins, as they do, for example, when forming a biofilm or in the lung of cystic fibrosis patients. In fact, if this were possible, the vaccination strategy would not be sensible, since it would result only in selection of a particular group of bacteria, rather than elimination of the bacteria.

For a successful vaccination, it must therefore be ensured that the pathogens entering the organism are unable to simply dispense with the corresponding surface protein or to not express it at all during part of their developmental cycle. The previous vaccines against bacteria have not always solved this problem to complete satisfaction. While most viruses are incapable of greatly altering their protein composition and while vaccinations against one or more of their surface proteins nearly always result in sufficient protection, bacteria are organisms which adapt to given environmental stimuli. The development of vaccines against bacteria is therefore very difficult, since it is not possible, without predicting the function, to reliably detect whether or not a special surface protein is indispensable for an infection.

It is therefore an object of the invention to provide means for being able to classify Pseudomonas aeruginosa strains with respect to their virulence factors and to influence them with respect to their risk potential. More specifically, it is the object to develop a strategy against Pseudomonas aeruginosa infections and to find possible targets for the development of vaccines, therapeutics and diagnostic agents.

The invention is based on the finding that various genes which are very different from one another are involved via their gene products in the virulence potential of Pseudomonas aeruginosa microorganisms and are virulence factors which are independent of one another but which partially complement and potentiate each other. The genes found within the scope of the present invention partly have completely different functions and are also located at completely different sites in the genome of the microorganism. It was not possible to predict their influence on virulence. “Switching off” one or more of the said genes impairs the virulence up to rendering said microorganism completely harmless.

The object of the present invention is achieved by:
finding novel virulence-determining genes in Pseudomonas aeruginosa;
using novel and known genes which have been found to be virulence-determining as targets for the development of medicaments and vaccines for blocking or switching off said genes or the corresponding gene products in vivo and also for the development of diagnostic agents;
using said virulence-determining genes for diagnosing virulent Pseudomonas aeruginosa strains.

The invention therefore comprises in detail:

1. Isolated or recombinant nucleic acids according to the sequences of Seq. ID 1, Seq. ID 2 and Seq. ID 3 or degenerated or modified sequences homologous thereto with corresponding function, said modifications comprising in particular deletions, insertions and/or substitutions of amino acids, in particular point mutations, truncations, extensions and the like, as long as said modifications do not substantially impair the overall function of the microorganism with respect to survivability;

2. Proteins encoded by any of the abovementioned sequences or by essentially functionally equivalent proteins or peptides homologous thereto, in particular those associated with sequence modification due to deletion, insertion or substitution of single and/or multiple amino acids, sequence-extending additions of single and/or multiple amino acids and/or chemical derivatization, in particular of the terminal amino acids.

3. The use of the nucleic acids or nucleotide sequences PA0740, PA1288, PA1322, PA1441, PA1572, PA1992, PA2591, PA3344, PA4621, PA5040, PA5349 and/or PA5415 of the microorganism Pseudomonas aeruginosa, denoted according to the nomenclature of the Pseudomonas aeruginosa Community Annotation Project (see www.pseudomonas.com), and/or of the nucleic acids or nucleic acid sequences according to Seq. ID 1, 2 or 3, or of the in each case corresponding proteins endogenous to Pseudomonas aeruginosa, all of which are essential for the survivability of said microorganism in humans or animals, as targets for the development of diagnostic agents for identifying the virulence of a Pseudomonas aeruginosa strain, for the development of therapeutics for Pseudomonas aeruginosa strains and for the development of vaccines against Pseudomonas aeruginosa strains.

It is also possible to use the sequences PA1104 and/or PA1452 instead of or in connection with PA 1441.

Usable proteins also comprise proteins homologous to said proteins or protein fragments of said proteins and homologous variants, in particular those associated with sequence modification due to deletion, insertion or substitution of single and/or multiple amino acids, sequence-extending additions of single and/or multiple amino acids and/or chemical derivatization, in particular of the terminal amino acids.

4. Antibodies directed against protein encoded by any of the nucleic acid sequences PA0740, PA1104, PA1288, PA1322, PA1441, PA1452, PA1572, PA1992, PA2591, PA3344, PA4621, PA5040, PA5349 and PA5415 of the microorganism Pseudomonas aeruginosa, denoted according to the nomenclature of the Pseudomonas aeruginosa Community Annotation Project, or of the nucleic acid sequences according to Seq. ID 1, 2 or 3 or at least one functional fragment of any of the abovementioned proteins for the use in determining the virulence of Pseudomonas aeruginosa strains.

5. Vaccines, comprising at least one protein encoded by any of the nucleic acid sequences PA0740, PA1104, PA1288, PA1322, PA1441, PA1452, PA1572, PA1992, PA2591, PA3344, PA4621, PA5040, PA5349 and PA5415 of the microorganism Pseudomonas aeruginosa, denoted according to the nomenclature of the Pseudomonas aeruginosa Community Annotation Project, or of the nucleic acid sequences according to Seq. ID 1, 2 or 3, or at least one functional fragment of any said proteins, or at least one fusion protein based on said protein or functional part of said protein, and
Vaccines, comprising at least one of the nucleic acids according to sequences PA0740, PA1104, PA1288, PA1322, PA1441, PA1452, PA1572, PA1992, PA2591, PA3344, PA4621, PA5040, PA5349 and PA5415 of the microorganism Pseudomonas aeruginosa, denoted according to the nomenclature of the Pseudomonas aeruginosa Community Annotation Project, or of the nucleic acids according to Seq. ID 1, 2 or 3 in a modified modification readable in mammalian cells and in relation to a promoter readable in mammalian cells.

In a development of the invention, the genes with the corresponding promoter may have been ligated into a plasmid. Furthermore, the vaccine may preferably comprise customary additives and excipients and also in particular at least one adjuvant.

The sequences Seq. ID 1, Seq. ID 2 and Seq. ID 3 are the following newly identified gene sequences:

DNA sequence:

Seq. ID 1

CTGCAGGGCTACGCTGAGGGAGTTGTAGAGGAGGCCGATCACAACACCCT
GCGCATCCGGCTAGCCCATGGCCTTCGAGTCAAAGAAGCGTCTCCCGCCG
CCGATGACGTGGCCGACGTTCCGCGTACGCCGGGGGTTAAGAAGCCTTCG
ATACGTCTGCTCGGGCTGCTCCAGCTGCTGTGGTTAGAGGCTGGACTGGC
CAACTGGTACCCACTGATGGAGGCAAGCGAACGCCCGCGAATGTTGCCTA
TTGGGTATCTGAAGCCGCAAAGCGTATCCGGGCCAGCCGAATGACGGTGT
TCGACGTCTTGCTGATGTCGGCCAAGAAGAACTCCCGCATGGCGAAGCGC
AATGCTGAGGTCGTGGAGCTGGCGCAGGAGCATTCGCGCAGGCTCATTGC
GGTGTCGCCGCTGGCGTCCTTCAACTCGGAACGACGCAATCTCTTGACGC
TTTCTGTCTCGGGACCCTTCGGGATGCCGACGATGGACATTCGAGCGGA

This DNA sequence is of crucial importance both for intracellular survival and for quorum sensing of P. aeruginosa. Said sequence is present in most, although not in all, pathogenic isolates of P. aeruginosa. Destruction of this sequence or of the gene product encoded thereby results in quorum sensing being switched off and thus in a reduction in pathogenicity.

DNA sequence:

Seq. ID 2

TGCAGGATCTGGGTCAGGTTGTCGATCAGGTTCTGGGTCAGCGAATTCAG
AGCTCTCATTCGTCCTGGCACTCCACTGCCGGTTGGTCTGGGAAATTAAT
TGGGAAGGGCGGCAGGCCGCGCGCCTTGTCGAGATCCGCTGCGACCTGCA
AGGCCGCCTCGAGCTCGTCGCAGCCGGTGGCTTGCATGATGTTGTAGCGC
TGGTTCTTTTCTTCGTTTTCGGTCATGGCCAGGGCCAGGTAGAGACTCGG
GGGPACCACACGGAAGAGGTACTCCTTGCCCTTGGCCAGGAGCACGCCCT
CGGTGAATTTGCCGCTTTCCTTGCGGGCCGAGAGCATCATCGACTTCTGC
GCCGGCGACAGCTCGCGGAACCTGGAGATCTTCTCTACCTCGTCTGGGGG
CATGTTCAGGCACAACCACCACTCGATCATGTTCAGCATCGGCGCCCCGG
AGGCTGGGATGTCGTCGATGTTCTGGGTGGCGAGCCAGAACCAGGCGCCC
AGTTTCCGCCACATCTTGGTGATCTTCATGGCGTAGGGCAGCAGCAGCGG
GTGCTTGGTGATGATGTGTCCCTCATCGGTGATCTTGACGATGGGCCGGC
CCTTGAATTGGTCGCGTTCGGGGATGTTGTTTACGGTGTTCAGCAGCGAG
ATGTAGGCGATCCCGAGCTGGGCGGCGTAGCCTTCGCGCGCGTAAGTCGC
GAAATCCACCACGGTGAGGTCGGCTTCAGGCCAGGGCGTGCCTTTGCGAT
TGAACATCTCGCC
DNA sequence:

Seq. ID 3

ATGCGTTGGAAACTCCCCTGGCCGAAGCTGGCCGCACCGAAGGTGGCCGC
GTCCGGCGCTGGCGATGACGAGCAGCCGGACGGCTGGCAGCGCCACGTCG
AGGCACTGCACCAGGCCGGCATCCCCGAACCCGGCACGGCGGTGCAAGGT
CACAGGCCGGCGACGATGGCAGACGAGCAGACGCTGTATGAGGTCGCGCC
GTCGTTCGTGGAACTACTGCCCTGGGTGGAGTTCCTGCCGCAGTCGAAGG
CCATGTTGCTGGAGGACGGGCAATCGGTGGCGGCCTTTTACGAGCTGGTG
CCGCTGGGCACCGAAGGCCGGGAACCCGGCTGGCTGGCGCAGGCCCGCGA
TGCCTTGGAGAACGCGCTGCAGGACAGTTTCGATGAACTGGACGAAACTC
CCTGGGTGTTGCAGCTCTACGCCCAGGACGAGCCCAGTTTTGACCAGTAC
ATGCAGACCCTGCGCGACTATGTGCAGCCGCGCGCCCGTGATACAGCGTT
CAGCGAGTTCTACCTGCGCTTCTTCGGCCACCACCTGCGCGCGGTGGCCA
AGCCGGGCGGCCTGTTCGAGGACACTGTGGTCACACGGCTGGGCTGGCGC
GGCCAGACCCGGCGCGTGCGCATGGTGGTCTACCGCCGCGTGACCGGGCA
AGGGCAAAACAGCCGTCGCGGGCAGACGCCCGAGCAGATGCTCAATATCG
TCTGCGACCGCCTGTGCGGCGGACTGGCGAACGCCGGCATTCAGGCCCGG
CGCATGGTCGCGGCAGATGTTCATGACTGGTTGCTGCGCTGGTTCAACCC
GCGCCCCACGTTGCTCGGGCCTGGGGCCGAAGACCGGGAGCGCTTCTATA
CATTGGCGCGCTATCCCGACAGTGCGGAGGAAGGCGAGGACGGCGAGATC
GAGCTGGCGAGCGGACGGGATTTCAGCCAACGGCTGTTCTTCGGCCAGCC
GCGTTCCGACGTGGCGCACGGCACCTGGCATTTCGACGGCATGCCGCACC
GCGTGCTGATCACCGACCGCCTGCGCATGCCGCCAGGCACGGGACACCTG
ACCGGCGAGACGCGCAAGGGCGACGCCATCAACACGTTGTTCGACCAGAT
GCCCGAGGACACCATCCTTTGCCTGACGCTGGTCGCCACGCCGCAGGATG
TTCTCGAAGCGGATCTCAATCATCTGGCGAAGAAGGCCGTGGGCGAAACC
CTGGCATCCGAGCAGACGCTCAAGGACGTGCATGAAGCCCGCTCCCTGAT
CGGCAGCGCGCACAAGCTCTATCGTGGCACGCTGGCGTTCTACCTGCGCG
GGCGCGACGAGGCGGAACTGGATCGGCGCGGCCTCGACCTGGCGAACGTC
ATGCTCAACGCCGGTTTGCAGCCGGTTCGCGAAGACGACGAGGTGGCACC
GCTGAACAGCTACTTGCGCTGGCTGCCGTGCTGCTACAACCCCGGCCAGG
ATCGGCGCAAGTGGTACACCCAACTGATGTTCGCCCAGCACGCGGCGAAC
CTCTCGCCCGTGTGGGGCCGCGCCCAAGGTACGGGGCACCCCGGCATCAC
GATGTTCAACCGCGGCGGCGGGCCGATCACCTTCGACCCGCTCPACCGCC
TGGATCGGCAGATGAATGCCCACCTGTTTCTGTTCGGCCCGACAGGTTCA
GGCAAGTCGGCGACCCTCAACAACCTCTTGAATCAGGTCACGGCGATCTA
CCGTCCCCGCCTGTTCATTGTCGAGGCCGGCAACAGCTTCGGCTTGTTGA
GCGACTTTGCCCGGCGCCTGGGCCTGACCGTGAATCGGGTCAAGCTCGCT
CCGGGCTCGGGCGTTACCCTGGCGCCGTTCGCGGATGCGCGCCGGCTGAT
CGAGACGCCCAGCGACGTGCAGACGCTCGACGCCGATGCGCTGGATGAAG
ACCTGCCTCCCGATGCTGTGGCCATGGAGGCAGATGAGCAGCGCGACGTA
CTGGGTGAACTGGAGATCACCGCGAGGCTGATGATCACCGGCGGCGAAGA
TAAAGAAGAAGCCCGGATGACGCGAGCCGACCGCTGACTGATCCGTCAGT
GCATCCTCGATGCCGCCGAGCACTGCCACAGCAAGGATGGCGAGAAGCGA
ACCGTTCTCACGCGCGATGTGCGCAACGCGCTGCGCACGCGCAGCCAGGA
CCCGACGCTGCCGGAAATGCGGCGTGTGCGACTGCTGGAGATGGCCGACG
CCATGGACATGTTCTGCCAAGGCACGGACGGCGAAATGTTCGACCGCGAC
GGTTCGCCGTGGCCCGAAGCCGACATCACCCTGGTGGATCTGGCGACCTA
TGCCCGCGAAGGCTACAACGCGCAGCTCTCTATTGCCTACATCAGCCTGA
TCAGCACGGTGAACAACATTGCCGAGCGCGATCAGTACCTGGGCCGCCCG
ATCATCAACGTCACCGACGAAGGACACATCATCACCAAGAACCCGCTGCT
CGCACCCTACGTGGTGAAGATCACCAAGATGTGGCGCAAGCTGGGGGCCT
GGTTCTGGCTCGCCACACAAAACATCGACGACTTGCCGCGCGCTGCAGAG
CCCATGCTCAACATGATCGAGTGGTGGATCTGCCTGTCGATGCCGCCCGA
TGAGGTGGAGAAGATCGCGCGGTTCCGCGAACTCTCGCGTGCGCAGAAGG
CGCTGATGCTTTCCGCGCGCAAGGAAGCCGGGAAGTTCACCGAGGGCGTC
ATCCTCTCCAAGAGCCTGGAAGTGCTGTTTCGGGCCGTGCCACCGAGCCT
CTATCTCGCGCTCGCGCAGACGGAACCCGAGGAGAAGGCCGAGCGTTACC
AGCTCATGCAGCAACACGGCATCAGCGAACTCGATGCGGGCTTCAAAGTG
GCCGAGAGAATCGACCAGGCGCGCGGCATCAAGTCGCCAGCCGTGGGCCT
GCCGCAATAG

The sequence denoted D8A6 (Seq. ID 2) is partly homologous to the ORF (open reading frame=that part of a gene, which is translated into a protein) denoted CB31 (Seq. ID 3). The latter is part of a highly homologous gene family which has not been described previously and which is present in most P. aeruginosa strains and other Gram-negative bacteria (Pseudomonas species such as P. syringae, P. fluorescens and genera such as Ralstonia, Burkholderia) in the same habitat. Said ORF encodes a protein which is essential for the quorum sensing function. Insertion of the transposon into D8A6 stopped production and release of homoserine lactones (the quorum sensing messenger molecules). A manifestation of a pathogenic phenotype is therefore no longer possible. In addition, high conservation of the proteins encoded by the sequences homologous to D8A6 allows expression of pathogenicity factors to be regulated across species boundaries. Thus, for example, D8A6/CB31 is one of the sequences required for making stable co-colonizations in the lung of a CF patient (here between P. aeruginosa and Burkholderia cepacia) possible. These stable multiple colonizations very often cause a rapid and fatal deterioration of the state of health of the affected patients.

By intervening in this regulatory chain (by therapy or vaccination), a distinct improvement in the clinical picture of co-colonized patients can be expected. In diagnostics, said genes are suitable for thus determining whether a pathogenic P. aeruginosa is capable of establishing in the patient a stable coexistence with other bacteria, which would increase its dangerousness.

For the purposes of the invention, the use of the gene PA1441 may be replaced or combined with the use of the genes PA1452 and/or PA1104, since these genes act together functionally (see PA1441).

Within the scope of the invention, P. aeruginosa mutants were studied for their survivability in neutrophilic granulocytes. The identified genes are absolutely required for P. aeruginosa in order to survive contact with said cells. Thus, bacteria which lack these genes can therefore be destroyed more readily by a cellular immune response. In addition, some of the conspicuous P. aeruginosa mutants also had increased sensitivity to the complement system of the blood serum.

The virulence is classified within the scope of the present invention in the following way:

The evaluation used is the quorum sensing and the survival of those Pseudomonas aeruginosa microorganisms in which a virulence gene has been inactivated in granulocytes. The classification is explained in more detail below.

It is now possible, by using the virulence genes found here which code for surface proteins as target of a vaccination strategy, to create for the bacteria in the vaccinated organism an environment to which they can no longer adapt. If said bacteria retain their surface proteins, they can be attacked by the antibodies, if, on the other hand, they dispense with said surface proteins, they are no longer capable of defending themselves against the neutrophilic granulocytes.

Besides from the use of some surface proteins as vaccines, said proteins may also serve as markers for estimating the pathogenic potential of a P. aeruginosa. In addition, it is also possible to use for such diagnostic studies intracellular proteins which are highly important for the pathogenicity and resistance to the immune response but which are themselves not accessible to antibodies, owing to their location. It is possible, via studies using ELISA or PCR, in which these special genes are addressed, to provide information on the pathogenic behavior of a bacterium (e.g. resistance to oxidative stress, ability to survive intracellularly, formation of biofilms, motility by means of flagella or pili, resistances to antibiotics) and thus to adjust, for example, the antibiotic therapy of a patient individually to the capabilities of the bacterium which has infected said patient.

The genes identified here are from the areas: resistance to oxidative stress, ability to survive intracellularly, formation of biofilms and motility by means of flagella or pili.

Gene Identification

The genes of the invention were identified with the aid of transposon mutagenesis. This involved first constructing a mutant library and determining whether or not the particular mutants were still virulent.

For those mutants in which quorum sensing and/or intracellular persistence had been modified or switched off, a subsequent functional analysis of the particular gene was carried out, so far as the function was not known.

Transposon Mutagenesis

Transposon mutagenesis involves introducing a transposon into a cell and integrating into the genome with the aid of a suitable vector system. To this end, a transposase cuts the genomic DNA sequence which then has protruding single strands and integrates the transposon which has blunt ends. A DNA polymerase fills in the resulting gaps so that the DNA sequence is duplicated to the left and to the right of the transposon insertion. The transposon Tn5 used in this work is inserted independently of the sequence with a sequence duplication of 9-12 bp. If the insertion takes place in a coding section, a gene is switched off and simultaneously labeled. If this loss causes a phenotypic alteration, then the relevant mutant can be selected. Starting from the known transposon sequence, the surrounding DNA region may be sequenced.

For irreversible transposon mutagenesis, a mini-Tn5 derivative in the vector pTnMod-OGm was used here (DENNIS & ZYLSTRA 1998).

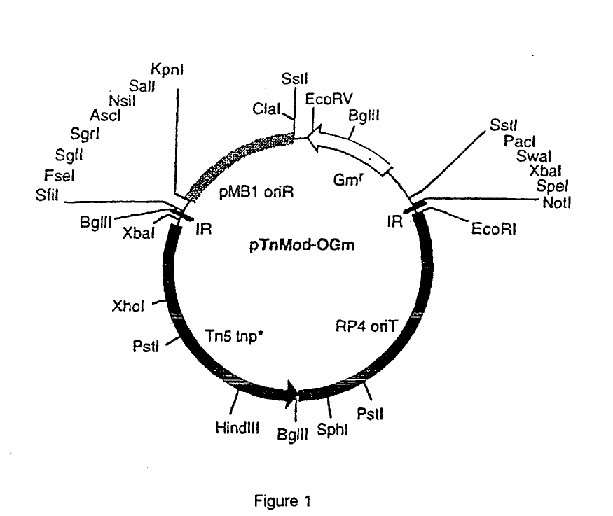

In pTnMod-OGm, the origin of replication, oriR PMB1, is located in the mini transposon so that, after genomic insertion, the remaining plasmid with the transposase can no longer be replicated. pTnMod-OGm is depicted in FIG. 1.

If then the resistance mediated by the transposon is selected for, any bacteria without transposon insertion are destroyed and only the mutants can grow.

If it is intended to check whether a gene switched off in this way is responsible for manifestation of a particular phenotype, then this can be carried out by comparing the original strain and the transposon mutant in a suitable differentiation approach. If the starting strain can survive a particular selection condition but the mutant cannot, then the switched-off gene is essential for the survival under these selected conditions.

Screening of the mutants may be facilitated with the aid of various known methods developed therefor. These include, inter alia, the signature-tagged mutagenesis (STM) method, the “in vivo expression technology (IVET) and methods utilizing DNA chip technology.

The STM method is described, for example, in
Hensel M, Shea J E, Gleeson C, Jones M D, Dalton E, Holden D W. Simultaneous identification of bacterial virulence genes by negative selection. (1995)
Hensel M. Whole genome scan for habitat-specific genes by signature-tagged mutagenesis. (1998)
Chiang S L, Mekalanos J J, Holden D W. In vivo genetic analysis of bacterial virulence. (1999)
Mecsas J. Use of signature-tagged mutagenesis in pathogenesis studies. (2002)

The in vivo expression technology (IVET) is a method for identifying any genes whose expression is increased under defined selection conditions (MAHAN ET AL. 1993; MAHAN ET AL. 1995; SLAUCH ET AL. 1994; RAINEY ET AL. 1997).

The application of DNA chip technology requires the genome of the organism to be studied to be completely sequenced. With this precondition, it is possible to construct a DNA chip to which the sequences of all putative ORFs are attached. If total mRNA is obtained from said organism, transcribed into cDNA fragments with the aid of random sequences and labeled appropriately, it is possible to construct by means of hybridization with the DNA chip an expression profile of the entire genome under the chosen growth conditions. Comparison of the expression profiles in various habitats makes it possible to identify any differences in the expression pattern (WEI ET AL. 2001).

Results of the Mutation Experiments and Allocation of a Function to the Genes Determined

Sequencing Reactions

The flanking regions of the transposon insertions introduced with the aid of plasmids were sequenced externally (industrial contract sequencing). The sequences obtained therefrom were examined for their occurrence in the already sequenced PAO genome (www.pseudomonas.com), using BlastN at the nucleotide level and BlastX at the protein level.

When the sequence was known, the respective promoter region of the identified gene was sequenced on both DNA strands in an overlapping manner. In the case that sequence variants causing an amino acid substitution of the encoded protein were additionally found in the DNA sequence of the HIT gene, said gene was amplified using PCR and again sequenced (Qiagen). Noncoding sequence variants were not checked any further. Confirmation of a nucleotide substitution was accepted only when the results of all sequencing reactions agreed.

In the case of unknown sequences, the NCBI database (www.ncbi.nlm.nih.gov) was screened for homologous sequences, using BlastN and BlastX.

The results of the sequencing reactions are intended to be illustrated here only briefly. The sequences themselves, unless present in the PAO genome, are depicted in the sequence listing. The results are interpreted in detail and evaluated below, separately for each transposon mutant. The numbering indicated of the genes, the percentage indicated of sequence identity and the descriptions refer to the information in the PAO database.

| | Comparison with PAO | | |
|---|---|---|---|
| | sequence | | |
| Gene | Identity | Sequence | |
| No. | (%) | variants | Description |
| PA0740 | 99.4 | *, (6) | homoserine lactonase |
| PA1104 | 98.0 | *, (8) | flil |
| PA1288 | 99.6 | (2) | fadL-, ompP1-homologous |
| PA1322 | 99.7 | (2) | pfuA |
| PA1441 | 99.6 | A-V, V-A, (1)P: 2 | homologous to fliK |
| PA1452 | 99.7 | (3) | flhA |
| PA1572 | 99.6 | T-A, *, (1) | hypothetical protein |
| PA1992 | 100 | | sensor protein |
| PA2591 | 100 | | transcription regulator |
| PA3344 | 99.4 | (3) | recQ |
| PA4621 | 99.8 | L-F, *, P: 1 | oxidoreductase |
| PA5040 | 99.4 | (3) | pilQ |
| PA5349 | 99.8 | (2) | rubredoxin reductase |
| PA5415 | 98.8 | G-A, R-Q, (8) | serine hydroxymethyl |
| | | | transferase |

Table 3.13. List of sequencing results.

The column “sequence variants” contains the particular amino acid substitutions in the protein (the first letter represents the PAO sequence, the second one represents the sequence found in P. aeruginosa TB). An “*” means that the gene has not been completely sequenced, due to its length. Therefore, there are possibly further sequence variants in the DNA sequence of this gene. The number in brackets indicates the number of noncoding nucleotide substitutions, the information after the abbreviation “P” represents mutations in the promoter region.

Apart from three unknown sequences without homologies in the databases, all of the other 14 DNA sequences of the PAO sequencing are known. The average sequence identity is 99.6%.

The present genes were identified by studying a large number of transposon mutants for their survivability in a particular habitat. The result obtained is information on which genes are essential for surviving under the chosen selection conditions.

The sequenced transposon mutants are listed together with the respective results of the in vitro and in silico studies below. In the individual case, further studies for phenotypical characterization of the mutants were carried out on the basis of these data. These results are likewise stated. The phenotypical properties of the particular mutant are described in more detail. Finally the available information of each transposon mutant is discussed in a summary.

In the case of those transposon mutants whose intracellular survival in granulocytes has been determined quantitatively, the list of phenotypical properties includes a quotient QAB. This is the quotient of the survivability in AB serum divided by the survivability in granulocytes. The value indicates, how much better the corresponding transposon mutant survived in the AB serum than in phagocyted form in granulocytes. P. aeruginosa mutants whose intracellular survival is impaired thus obtain values which are distinctly greater than one. Bacteria which, in contrast, are sensitive to serum but continue to be able to persist well in granulocytes have values of less than/equal to one.

PA0740

Phenotypical Properties

| | |
|---|---|
| quorum sensing | increased |
| further properties | increased protease secretion |

Switched-Off Gene (According to P. aeruginosa Genome Project)

| | |
|---|---|
| gene number | PA0740 |
| name | yjcS |
| function | lactonase, previously |
| | assumed: β-lactamase |
| structural features/ | 62% similarity to |
| homologies | hypothetical |
| | protein YjcS from |
| | E. coli |
| | structural features of |
| | the metallo-β-lactamase |
| | superfamily |
| nucleotide substitutions | synonymous: 452T-C; |
| | 545T-G; 551T-C; 893C-T; |
| | 908T-C; 950T-C |
| length | 1977 bp = 658 aa. |

Influence on Neighboring Genes

All genes in the vicinity of PA0740 are encoded on the other DNA strand. PA0740 itself has a typical promoter and terminator structure. Cis effects can thus be ruled out.

Further Studies

Determination of the minimal inhibitory concentrations (MIC) of various β-lactam antibiotics revealed no increased sensitivity of the transposon mutant compared to the wildtype. Examination of the culture supernatant showed that the homoserine lactone concentration is likely to be increased in a mutant having a transposon inserted in PA0740.

Summary

In contrast to the annotation in the genome project, PA0740 is not a β-lactamase but a homoserine lactonase required by P. aeruginosa in order to attach the homoserine lactones produced when switching to the stationary growth phase (a substrate of the class of β-lactams was not found for PA0740). Considering the homology of β-lactamases whose function is cleavage of the ring system of β-lactam antibiotics, it can be assumed that PA0740 is a homoserine lactonase which catalyzes a first step in the degradation of homoserine lactones by cleaving their ring structure. Switching off this gene results in a distinct increase in the concentration of homoserine lactones in the medium and thus in an increased response of the bacteria to this signal, for example a distinct increased secretion of proteases.

PA1288

Phenotypic Properties

| | |
|---|---|
| survivability in granulocytes | 0.07 |
| in AB serum | 0.8 |
| Q_{AB} | 11 |
| quorum sensing | switched off |
| further properties | no protease |
| | secretion, |
| | increased sensitivity |
| | to peroxides |

Switched-Off Gene (According to P. aeruginosa Genome Project)

| | |
|---|---|
| gene number | PA1288 |
| function | possible transport |
| | protein of the outer |
| | membrane |
| structural features/homologies | 47% similarity to |
| | fadL (transporter for |
| | long-chain fatty |
| | acids in the outer |
| | membrane of E. coli) |
| | 43% similarity to |
| | ompP1 of Haemophilus |
| | influenzae (possible |
| | virulence factor) |
| nucleotide substitutions | synonymous: 638C-T; |
| | 935C-T |
| length | 1275 bp = 425 aa. |

Influence on Neighboring Genes

Downstream of PA1288, there is a distinct termination loop. The next three genes are encoded on the opposite strand. A direct influence of the transposon insertion on the surrounding genes is therefore very unlikely.

Further Studies

The stress to which the phagocyted bacteria are exposed in granulocytes is mainly oxidative. To simulate these conditions, an appropriate transposon mutant 14C5 was streaked out on LB agar containing different concentrations of hydrogen peroxide and incubated at 37° C. for 16 hours. Thus, the stress exerted on said bacteria did also not last over the entire incubation period but acted only initially for a relatively short time. P. aeruginosa which were not destroyed in this initial phase were able to grow in an undisturbed manner thereafter.

The resistance to hydrogen peroxide was found to be distinctly limited for the transposon mutants having insertions in PA1288, PA3344, PA4621 and PA5349, in comparison with the wildtype.

Further Information

Two genes (PA4589 and PA1764) paralogous to PA1288 exist in the P. aeruginosa PAO genome, both of which are listed in the PAO database as possible membrane proteins of the outer bacterial membrane. Further information was obtainable in the databases only on fadL and ompP1.

fadL:

FadL transports long-chain fatty acids through the outer cell membrane. A second enzyme (fatty acid acyl-CoA synthetase (FACS)) then transports said fatty acids through the inner cell membrane and, after activation with CoA, introduces them to β-oxidation, with FadL determining uptake specificity and FACS rendering the uptake process irreversible (DIRUSSO CC, BLACK PN, 1999). The FadL structure corresponds to a β-barrel of 20 antiparallel strands whose amino acid sequence determines the specificity of the channel thus formed. FadL is expressed especially during the stationary E. coli growth phase. Bacteria in which FadL has been switched off due to mutation exhibit only a small change in protein composition during transition from the logarithmic to the stationary phase and survive with limited nutrient supply only for a short time (FAREWELL A ET AL. 1996).

ompP1:

OmpP1 is a protein of the Haemophilus influenzae outer cell membrane. (BOLDUC GR 2000). Various variants of said protein exist in H. influenzae. The protein is expressed during infection of a host organism and its expression is not downregulated, even in the case of a specific immune response to its exterior epitopes. OmpP1 is therefore suitable in principle for immunization against H. influenzae.

Summary

PA1228 codes for a protein which corresponds to a β-barrel with respect to its structure. The specificity of the channel thus formed remains unknown and the recognized substrate is presumably hydrophobic (aliphatic or aromatic). On the basis of the orthologous genes described in the literature, the corresponding substrate can be assumed to be activated with coenzyme A and metabolized inside the bacterium (e.g. β-oxidation).

From the studies carried out, PA1288 functionality was known to be essential for production of aliphatic homoserine lactones and for intracellular survival in granulocytes. These observations are confirmed by the results of studies in E. coli and H. influenzae, described in the literature (see above). The exact function of said protein in P. aeruginosa is not known but the orthologous E. coli protein FadL has been studied in detail. FadL transports in E. coli long-chain fatty acids through the outer cell membrane. Expression of this gene is distinctly increased with external stress (high growth density or incubation at 42° C.). Switching off fadL results in the bacteria no longer being able to switch from logarithmic growth to the stationary phase. These mutants likewise survive only poorly phases of starvation. Haemophilus influenzae requires a corresponding orthologous gene for infection. These findings lead to the conclusion that in P. aeruginosa too, the function of the FadL-like protein is somehow linked to the reaction to external stress factors.

It is not possible to explain beyond doubt, how PA1288 exerts influence on autoinducer production or intracellular stability. One hypothesis would be that PA1288 is essentially required for uptake of long-chain aliphatic compounds which, after further processing, are needed as acyl side chains in the production of aliphatic homoserine lactones (AHL). This would also explain why the corresponding gene in E. coli is upregulated in the stationary phase. AHL production and thus also the need for aliphatic compounds for synthesizing the homoserine lactone side chain are at a maximum under these conditions. PA1288 knock-out could thus block AHL synthesis in P. aeruginosa.

The transposon mutants were no longer capable of responding adequately to various types of external stress: intracellularly, in granulocytes, the bacteria were unable to survive oxidative stress and, when studying quorum sensing, an increased production of homoserine lactones, the adequate response to stress due to high growth density, was no longer observable. This can be compared to the response of E. coli to switching off fadL.

Switching to stationary phase conditions causes the P. aeruginosa wildtype to upregulate likewise defense mechanisms against external stress. In the study of intracellular survivability, bacteria of this growth phase were transferred into the granulocytes. However, when the transposon mutants having an insertion in PA1288 did not adjust to the stationary phase conditions, this being indicated by low production of pyoverdin and homoserine lactones, then said mutants were much more susceptible to external stress and thus unable to survive in the granulocytes. This agrees with the finding that P. aeruginosa has much stronger defense mechanisms against the immune response of a host in the stationary phase than in its logarithmic growth phase.

PA1322

Phenotypic Properties

| | |
|---|---|
| quorum sensing | switched off |
| further properties | no protease secretion, |
| | increased resistance to |
| | peroxide |

Switched-Off Gene (According to P. aeruginosa Genome Project)

| | |
|---|---|
| gene number | PA1322 |
| name | pfuA |
| function | possible TonB-dependent |
| | receptor |
| structural features/ | 44% similarity to a |
| homologies | ferrichrome iron receptor |
| | (S. paratyphi) |
| | C-terminal TonB-receptor |
| | cassette |
| nucleotide substitutions | synonymous: 1559G-A; 1613G- |
| | A) |
| length | 2199 bp = 732 aa. |

Influence on Neighboring Genes

A strong termination sequence is located downstream of pfuA. Even during sequencing, big problems were encountered in this region. Thus, an effect of transposon insertion on transcription of the downstream genes cannot be ruled out.

Further Studies

An insertion in PA1322 resulted in increased resistance to peroxides.

Further Information

TonB is a secreted protein that binds with high affinity and delivers to P. aeruginosa iron from the surrounding medium. If TonB is switched off in P. aeruginosa PAO, then pyoverdin and pyochelin are also no longer produced. Likewise, P. aeruginosa PAO mutants having a defective tonB are, in contrast to the wildtype, no longer capable of lethally damaging immunosuppressed mice or even of just surviving. The TonB-dependent iron uptake is thus essential for P. aeruginosa in order to infect a host (TAKASE H ET AL. 2000)

Summary

The current literature describes P. aeruginosa iron uptake being regulated via quorum sensing. In the transposon mutants found here, however, regulation proceeds in the opposite direction: switching off a TonB-dependent iron receptor causes homoserine lactone production to be switched off. A regulatory mechanism of this kind has not been described in the literature previously. In a different context, however, inactivation of TonB revealed that this mutant likewise no longer produces pyoverdin. However, production of these siderophores is directly regulated via quorum sensing so that here too, a connection between iron uptake and quorum sensing expression was detected. The results of the studies in the present work prove beyond doubt the direction of the regulation found here in which PfuA has a regulatory influence on quorum sensing.

PA1441, (PA1452, PA1104)

Phenotypic Properties

| | | |
|---|---|---|
| survivability | in granulocytes | 0.08 |
| | in AB serum | 0.7 |
| | Q_{AB} | 9 |

| | |
|---|---|
| further properties | the mutant appears to be |
| | immobile in microscopic |
| | observations and exhibited |
| | virtually no invasivity in |
| | invasivity assays using |
| | epithelial cells. |
| | Truncated, structurally |
| | altered flagella are |
| | visible in electron- |
| | microscopic images. |

Switched-Off Gene (According to P. aeruginosa Genome Project)

| | |
|---|---|
| gene number | PA1441 |
| function | hypothetical, unclassified |
| | protein |
| | presumably flagella |
| | construction |
| structural features/ | 47% similarity to fliK |
| homologies | (S. typhimurium) |
| nucleotide substitutions | nonsynonymous: GCC GTC: |
| | A74V |
| | GTC-GCC: V82A |
| | synonymous: 164G-A; 776C-T |
| | promoter: -82C-G; -29G-A |
| length | 1284 bp = 427 aa. |

Influence on Neighboring Genes

A distinct termination structure adjoins PA1441 downstream. Other genes are therefore unlikely to be transcribed together with PA1441.

Further Genes

Switching off the genes PA1452 (flhA) or PLA1104 (Flil) likewise produced mutants which have reduced survivability in granulocytes.

Further Information

The construction of flagella was studied in detail on S. typhimurium and E. coli by way of example. According to this, the genes required for flagella synthesis are arranged in three large adjoining operons. Only after all of the proteins encoded in one operon have been completely synthesized and incorporated according to their function into the nascent flagellum, is the next operon read. fliK is the last gene of the second operon, with which synthesis of the basal plate of the flagellum is completed. In this connection, S. typhimurium FliK has at least two functions. First, it determines the specificity of the central channel of the basal plate and regulates, whether proteins for the flagellar hook or flagellin itself are exported (MACNAB 1992). Secondly, it functions as a chaperone for the proteins passed through the channel. If fliK is switched off in S. typhimurium, the mutants are immobile and either have no flagella or have, instead of correctly assembled flagella, functionless extended flagellar hooks (poly hooks) which have a corkscrew-like structure.

The abovementioned operon structure can be found in principle also in P. aeruginosa. However, the last gene of the second operon (fliK) is separated by a 372 kbp insertion from the rest of the operon which now ends in fliJ. fliK itself is still directly located upstream of the 3^{rd} operon for flagella synthesis but is separated from the latter by a distinct termination loop.

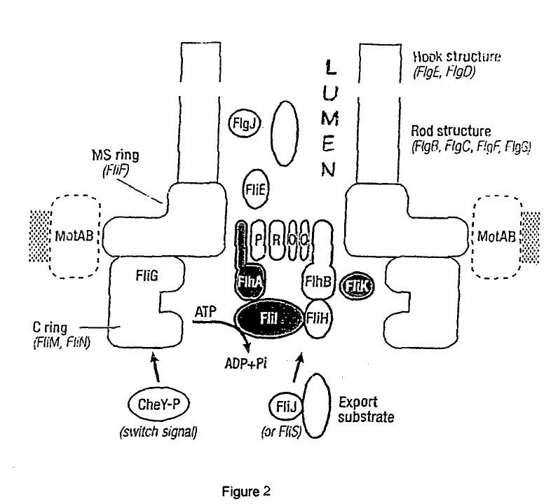

FliI and FlhA constitute together with FliK necessary components of the flagella export system. FlhA is a structural component, FliI enables the particular proteins to be exported with ATP consumption (MINAMINO, MACNAB, 1999). FIG. 2 depicts the location of FliK, FliI and FlhA in the flagellum.

FIG. 2 depicts the P. aeruginosa secretion system. The genes in question are marked in dark gray in the picture. All three genes addressed here are required for the secretion of pathogenicity factors through the flagellum. All of them have been proven necessary for the intracellular survival of P. aeruginosa.

Further Studies

In the studies on invasivity in epithelial cells (chapter 3.5.1.), mutants having a transposon insertion in fliK exhibited the lowest invasivity. Observation under the microscope likewise revealed their complete immobility. Electron-microscopic images of their flagella were therefore produced for a more detailed investigation. These images showed that approx. 90% of PA1441 transposon mutants have distinctly truncated flagella, the remaining mutants having no flagellum at all. Switching off fliK in S. typhimurium results in the formation of extended hook structures. In order to be able to decide, whether the observed structures are truncated flagella or extended hooks, the flagellins of fixed bacteria (P. aeruginosa TB_{wt}, P. aeruginosa PA1441-knock out and E. coli DH5α or DH5a were detected with a specific anti-flagellin antibody (against type b flagellin).

FlhA mutants have no flagella and, although fliI mutants have flagella which are functional in principle, the latter are not tightly integrated in the membrane and are rapidly lost together with the connected export system.

Summary

Expression of PA1441 is essential for P. aeruginosa motility. In contrast to E. coli and S. typhimurium, however, a truncated flagellum continues to be synthesized when said gene is switched off. In P. aeruginosa, fliK is separated from the rest of the second operon for flagella synthesis by a large insertion. The reduction in intracellular survivability of P. aeruginosa by switching off the genes PA1104, PA1441 and PA1452 suggests that the flagellar export system is essential for the survival of said bacteria in phagocytes. This corresponds to the experience from Yersinia pseudotuberculosis, another pathogen capable of intracellular survival in which the proteins secreted by the flagellar export system are essential for intracellular persistence (YOUNG ET AL., 1999). Since the export systems of Pseudomonas and Yersinia are highly homologous, a function of a similar kind in the secretion of pathogenicity factors is an adequate explanation of the necessity of said genes for survival in granulocytes.

PA1572

Phenotypic Properties

| | | |
|---|---|---|
| survivability | in granulocytes | 0.06 |
| | in AB serum | 1.2 |
| | Q_{AB} | 20 |

| | |
|---|---|
| quorum sensing | reduced production of |
| | autoinducers |
| further properties | reduced protease secretion |

Switched-Off Gene (According to P. aeruginosa Genome Project)

| | |
|---|---|
| gene number | PA1572 |
| function | hypothetical protein, |
| | unclassified |
| structural features/ | 56% similarity to a |
| homologies | hypothetical protein of |
| | 377 aa in length in |
| | Pyrococcus horikoshii |
| nucleotide substitutions | nonsynonymous: ACC-GCC: |
| | T354A |
| | synonymous: 1007C-G |
| Length | 1146 bp = 382 aa. |

Influence on Neighboring Genes

The last 20 bases of the coding sequence of PA1572 form together with the downstream sequence a typical termination structure. It cannot be assumed therefore that other genes are transcribed together with PA1572.

Further Studies

It was shown that incubation with P. aeruginosa TB_{wt} was able to increase the reduced secretion of proteases and homoserine lactones almost to the starting levels of the wildtype.

Summary

In the transposon mutant having an insertion in PA1572, the switched-off gene is unclassified so that neither binding partner nor substrates are known. Nevertheless, said gene is one of the most interesting genes found in these studies. While serum resistance is unchanged, possibly even somewhat increased, compared to the wildtype, almost no PA1572-knock out mutants survive phagocytosis by granulocytes. They likewise exhibit greatly reduced production of homoserine lactones but no complete stop. It was possible, by adding autoinducers, to induce in mutants having an insertion in PA1572 endogenous production of said molecules. This leads to the conclusion that transposon insertion has influenced neither uptake nor recognition or production of homoserine lactones but the regulation thereof.

PA1992

Phenotypic Properties

| | | |
|---|---|---|
| survivability | in granulocytes | 0.4 |
| | in AB serum | 0.4 |
| | Q_{AB} | 1 |

| | |
|---|---|
| further properties | distinct increased |
| | invasivity into epithelial |
| | cells and increased |
| | adherence. |

Switched-Off Gene (According to P. aeruginosa Genome Project)

| | |
|---|---|
| gene number | PA1992 |
| function | possible 2-component sensor |
| structural features/ | 56% similarity to |
| homologies | unpublished Paracoccus |
| | denitrificans flhS (only C- |
| | terminal 75% of ORF) |
| | structural motifs of a |
| | histidine kinase and a |
| | response regulator receiver |
| nucleotide substitutions | nonsynonymous: CTG- |
| | TTG: L326F |
| | in the promoter: -75 C-T |
| length | 1694 bp = 565 aa. |

Influence on Neighboring Genes

PA1992 is the last gene of a possible polycistronic gene cassette which ends without termination loop; however, the genes on the downstream 8 kb are encoded on the opposite strand. A cis effect of transposon insertion is thus unlikely.

Further Information

Two genes paralogous to PA1992 exist in the PAO genome. PA1976 is slightly longer, but has some very homologous regions. The second paralogous gene is PA3271. Both genes are functionally classified as 2-component sensors. The databases yield no further information on the function of PA1992 or flhS.

Further Studies

When studying intracellular survival in granulocytes, the mutant 14B2 usually exhibited a survival rate which was distinctly above the average survival rate. However, the results of the invasivity assays with epithelial cells were especially conspicuous (chapter 3.5.1.). In these, the transposon mutant exhibited a distinctly higher adherence and 10-20 times higher invasivity in epithelial cells than the wildtype.

Summary

PA1992 is a histidine kinase. This structural motif indicates a function as regulator in a signal cascade. Switching off the PA1992 gene increased the adherence and invasivity into epithelial cells by at least a factor of 10. The genomic vicinity does not allow for a cis effect. The phenotypic alterations must therefore be directly attributed to switching off the PA1992 gene.

PA2591

Phenotypic Properties

| | |
|---|---|
| quorum sensing | switched off |
| further properties | no protease secretion |

Switched-Off Gene (According to P. aeruginosa Genome Project)

| | |
|---|---|
| gene number | PA2591 |
| function | transcription regulator |
| structural features/ | 46% similarity to DMSO- |
| homologies | reductase regulator protein |
| | DorX (Rhodobacter |
| | sphaeroides) signature of |
| | the LuxR family |
| length | 807 bp = 268 aa. |

Influence on Neighboring Genes

A possible termination loop is located downstream of PA2591. However, this structure is not particularly well-developed. Therefore, the downstream genes PA2590 and PA2589 (both hypothetical ORFs) are possibly also transcribed by the RNA polymerase.

Summary

The identified PA2591 gene belongs to the family of LuxR regulators, as does Vfr, a known quorum sensing regulator, for example. In all of those Gram-negative bacteria whose quorum sensing has been studied previously, at least one LuxR-related protein is part of the quorum sensing regulatory system. In this context, switching off PA2591 interestingly resulted in a complete failure of quorum sensing in P. aeruginosa TB, meaning that this regulator also intervenes in quorum sensing control. Previously, the only known regulator on a higher level was Vfr. The function of the LuxR regulator found was previously unknown. The results of the studies showed that transposon insertion into this gene was unable to cause the transposon mutant to produce either short-chain or long-chain aliphatic homoserine lactones. This means that this previously functionally uncharacterized regulator intervenes in autoinducer production in a similarly central position as Vfr, but has previously been overlooked.

PA3344

Phenotypic Properties

| | | |
|---|---|---|
| survivability | in granulocytes | 0.05 |
| | in AB serum | 0.9 |
| | Q_{AB} | 18 |

| | |
|---|---|
| further properties | increased sensitivity to |
| | peroxides |

Switched-Off Gene (According to P. aeruginosa Genome Project)

| | |
|---|---|
| gene number | PA3344 |
| name | recQ |
| function | ATP-dependent DNA helicase |
| structural features/ | DEAD-box subfamily of |
| homologies | ATP-dependent helicases, |
| | conserved C terminus |
| | typical for helicases, HRDC |
| | domain |
| nucleotide substitutions | synonymous: 95A-C; 337A-G; |
| | 382C-T |
| length | 2138 bp = 713 aa. |

Influence on Neighboring Genes

Another 4 genes of unknown function are located directly downstream of recQ in the same reading orientation on the PAO chromosome. A cis effect would be theoretically conceivable; the results of continuing studies, however, make recQ appear to be actually responsible for the observed phenotype.

Further Information

The functional prediction obtained from the PAO database reads: DNA replication, DNA recombination, DNA modification and DNA repair.

Further Studies

A transposon mutant having an insertion in recQ exhibits no substantially altered phenotype under normal growth conditions or when incubated with AB serum. However, switching off RecQ results in a substantial reduction in intracellular survivability in the granulocytes and in a distinctly increased sensitivity to peroxides. The function of this protein must therefore be the repair of DNA damage induced under these circumstances.

Summary

AB serum did not damage the mutants any more than the wildtype. Only the ability to survive intracellularly after phagocytosis by granulocytes was greatly reduced. Switching off recQ increased sensitivity to oxidative stress in the granulocytes. This has been confirmed by the lack of growth on peroxide-containing complete medium. Presumably, recQ is involved in the repair of damage to the DNA caused by oxidative stress.

PA4621

Phenotypic Properties

| | | |
|---|---|---|
| survivability | in granulocytes | 0.2 |
| | in AB serum | 0.2 |
| | Q_{AB} | 1 |

| | |
|---|---|
| quorum sensing | switched off |
| further properties | no protease secretion, |
| | increased sensitivity to |
| | peroxides |

Switched-Off Gene (According to P. aeruginosa Genome Project)

| | |
|---|---|
| gene number | PA4621 |
| function | possible oxidoreductase |
| structural features/ | C-terminal aldehyde |
| homologies | oxidase and xanthine |
| | dehydrogenase motifs, 2 |
| | transmembrane helices |
| | predicted |
| nucleotide substitutions | nonsynonymous: CTG- |
| | TTG: L326F |
| | in the promoter: -75 C-T |
| length | 2832 bp = 944 aa. |

Influence on Neighboring Genes

There is no termination structure downstream of PA4621. The gene forms together with the three downstream genes PA4620 (4-hydroxybenzoyl-CoA reductase), PA4619 (membrane-bound alcohol dehydrogenase cytochrome c subunit) and PA4618 (unknown function) a polycistronic gene cassette.

Summary

The mutant having an insertion in PA4621 is distinguished by a defective quorum sensing and reduced survivability in AB serum, thereby becoming conspicuous in the selection experiments with granulocytes. The latter can be attributed to an increased sensitivity to oxidative stress in the phagosomes. The information in the databases on the switched-off gene contains no information on a possible substrate or further experimental results for the encoded protein.

PA5040

Phenotypic Properties

| | | |
|---|---|---|
| survivability | in granulocytes | 1.0 |
| | in AB serum | 2.7 |
| | Q_{AB} | 2.7 |

| | |
|---|---|
| further properties | best of all transposon |
| | mutants studied in |
| | surviving incubations with |
| | granulocytes. |

Switched-Off Gene (According to P. aeruginosa Genome Project)

| | |
|---|---|
| gene number | PA5040 |
| name | pilQ |
| function | possible oxidoreductase |
| structural features/ | required for type IV |
| homologies | fimbria construction. Forms |
| | a basal component through |
| | which the Pili proteins are |
| | exported. Type IV fimbria |
| | are necessary for motility |
| | (twitching motility) and |
| | for contact with surfaces |
| | (HOBBS & MATTICK 1993) |
| nucleotide substitutions | synonymous: 488C-T; 743C-T; |
| | 803T-C |
| length | 2145 bp = 714 aa. |

Influence on Neighboring Genes

pilQ is the last gene of the polycistronic gene cassette pilMNOPQ. Further genes whose function is in the amino acid metabolism (aromatic amino acids) or in heme synthesis are encoded directly downstream, without obvious termination structures.

Further Studies

The transposon mutant in which the transposon was inserted into the pilQ (PA1322) has a distinctly increased serum stability. PilQ is a component of P. aeruginosa type IV fimbria (MARTIN ET AL. 1993).

Summary

The high rate of survival of pilQ transposon mutants can be attributed primarily to increased resistance to blood serum. The genes for amino acid or heme synthesis which may be read together with pilQ by RNA polymerases have no decisive influence on the serum stability of bacteria. The observed alterations in the phenotype must therefore be attributed to the absence of the corresponding gene product. Increased production of exopolysaccharides which shield the bacteria from the complement system seems to compensate for the switching-off of PilQ.

PA5349

Phenotypic Properties

| | | |
|---|---|---|
| survivability | in granulocytes | 0.05 |
| | in AB serum | 0.4 |
| | Q_{AB} | 8 |

| | |
|---|---|
| quorum sensing | switched off |
| further properties | no protease secretion, |
| | increased sensitivity to |
| | peroxides |

Switched-Off Gene (According to P. aeruginosa Genome Project)

| | |
|---|---|
| gene number | PA5349 |
| function | possible rubredoxin |
| | reductase |
| structural features/ | 59% similarity to |
| homologies | rubredoxin reductase of |
| | Acinetobacter calcoa |
| | ceticus partial signature |
| | of an aromatic hydroxylase |
| | (flavoprotein |
| | monooxigenase), |
| | structural feature of a |
| | pyridine nucleotide |
| | disulfide oxidoreductase |
| nucleotide substitutions | synonymous: 893G-C; 1055T-C |
| length | 1155 bp = 384 aa. |

Influence on Neighboring Genes

The genes upstream of PA5349 code for various enzymes of the carbon catabolism. PA5350 and PA5351 for two rubredoxin ORFs, the gene (PA5348) downstream of PA5349 codes for a possible DNA-binding protein to which (in the databases) a function in DNA replication, DNA modification, DNA recombination or DNA repair is assigned. All of these genes (from PA5355 to 5347) are read by RNA polymerase presumably in one go.

Further Information

Rubredoxin reductase is a flavoprotein oxidoreductase. It operates together with rubredoxin and thus oxidizes aliphatic hydrocarbons with oxygen to give the corresponding carboxylic acid which can then be metabolized further by other enzymes. However, rubredoxin reductase still has also another function: it additionally protects some anaerobic bacteria from oxidative stress (LUMPPIO HL ET AL., 2001). In bacteria with aerobic metabolism, rubredoxin and rubredoxin reductase may also be an important protection against oxidative stress. Thus, for example, they can replace superoxide dismutase in E. coli (PIANZZOLA M J ET AL. 1996).

In P. aeruginosa, expression of two superoxide dismutases and a catalase is regulated directly via the LasR/LasI system. This bacterium too, thus requires quorum sensing for controlling oxidative stress. Mutants in which either of the two regulatory systems is defective, exhibit reduced resistance to hydrogen peroxide (HASSETT DJ ET AL. 1999), as was observed here too, when rubredoxin reductase was switched off.

Further Studies

Corresponding to the studies on other mutants, the mutant having a transposon insertion in PA5349 was also checked for its sensitivity to peroxides. This mutant too, is distinguished by markedly reduced resistance to oxidative stress.

Summary

The transposon mutant having an insertion in PA5349 showed markedly reduced survivability when incubated with granulocytes. Similar to other mutants having a defective helicase this may be attributed directly to reduced resistance to oxidative stress, on the basis of the results of incubation on peroxide-containing complete medium. In P. aeruginosa, at least two superoxide dismutases and a catalase are known to be involved in resistance to hydrogen peroxide in the stationary phase (HASSETT D J ET AL. 1999). The functionally very similar rubredoxin reductase could also possibly be classified into this category. Detoxification of oxygen is crucially important for P. aeruginosa. The failure of any of the detoxification mechanisms may possibly be the reason for phagocyted mutants having a defective PA5349 being no longer able to survive intracellularly in granulocytes.

Furthermore, the mutant having the insertion in PA5349, did not produce any homoserine lactones. Presumably, rubredoxin reductase is required in order to oxidize aliphatic hydrophobic compounds in such a way that they can be processed in the fatty acid metabolism. Failure of this enzyme could influence the synthesis of aliphatic homoserine lactones, since there are, due to the knock out, only a few, if any, aliphatic side chains for AHL synthesis present in the mutants (see also PA1288).

PA5415

Phenotypic Properties

| | |
|---|---|
| quorum sensing | no production of short- |
| | chain AHL, only very low |
| | production of long-chain |
| | AHL. |
| further properties | protease secretion not |
| | switched off |

Switched-Off Gene (According to P. aeruginosa Genome Project)

| | |
|---|---|
| gene number | PA5415 |
| function | serine hydroxymethyl |
| | transferase (amino acid |
| | biosynthesis and amino acid |
| | metabolism: Gly, Ser, Thr |
| | metabolism, Lys synthesis, |
| | CH₄ metabolism and C₁ |
| | reservoir) |
| structural features/ | 82% similarity to E. coli |
| homologies | serine hydroxymethyl |
| | transferase. Structural |
| | motifs of a class II |
| | aminotransferase and a |
| | binding site for pyridoxal |
| | phosphate |
| nucleotide substitutions | nonsynonymous: CGG-CAG: |
| | R76Q GGG-GCC: G304A |
| | synonymous: 161C-T; 228G- |
| | A; 251T-C; 287G-A; 308A-G; |
| | 416A-C; 770T-C; 935T-C |
| length | 1254 bp = 417 aa. |

Influence on Neigboring Genes

At least 4 different possibilities of forming termination structures exist downstream of PA5415. They are possibly protein binding sites by which transcription and translation of the downstream genes (sarcosine oxidase and proteins of the C₁ metabolism) can be regulated.

Summary

A connection, as regards contents, of serine hydroxymethyl transferase to quorum sensing cannot be found in the current literature. However, there exist results from E. coli, according to which these bacteria change their expression pattern under stress. In this case, production of serine hydroxymethyl transferase together with that of the protein FadL and the acyl carrier protein is markedly increased (OHBA ET AL. 1997). In the course of the studies, one mutant, PA1288, having the transposon insertion in a protein corresponding to FadL was found. This mutant too, is defective in quorum sensing and in the response to stress factors. The only functional connection known is serine hydroxymethyl transferase having a function in the C1 metabolism. S-adenosyl methionine is an internal reservoir for the C1 metabolism but, on the other hand, is also required for producing homoserine lactones. Switching off serine hydroxymethyl transferase possibly causes a reduction in the concentration of available S-adenosyl methionine and thus in a lack of homoserine for autoinducer synthesis.

Seq. ID 1

Phenotypic Properties

| | | |
|---|---|---|
| survivability | in granulocytes | 0.3 |
| | in AB serum | 0.15 |
| | Q_{AB} | 0.5 |

| | |
|---|---|
| quorum sensing | switched off |
| further properties | no protease secretion |

Switched-Off Gene (According to P. aeruginosa Genome Project)

| | |
|---|---|
| gene number | not present in PAO genome |

Further Information

There is low similarity (2·e⁻¹⁹) between the sequenced region and a hypothetical Salmonella typhi ORF at the protein level. There were no homologies at the nucleotide level.

Further Studies

The PstI-digested genomic DNA of various P. aeruginosa isolates was hybridized with the sequence flanking the transposon insertion in a Southern hybridization. Seq. ID1 is present not only in P. aeruginosa TB but also in the two isolates studied, P. aeruginosa CSGB8 and SG17M. As expected, P. aeruginosa PAO gave no signal.

Summary

The unknown sequence is no clone-specific DNA of P. aeruginosa TB, since it occurs also in two isolates of clone C (P. aeruginosa SG17M and CSGB8). A transposon insertion in this region which does not exist in P. aeruginosa PAO completely switched off P. aeruginosa TB quorum sensing, meaning that quorum sensing in these bacteria is regulated partly in a different manner. Moreover, the unknown DNA sequence, at least in a P. aeruginosa subpopulation, encodes additional crucial factors for quorum sensing expression which are not found in studies of the genetic reference strain PAO.

Seq. ID 2

Phenotypic Properties

| | |
|---|---|
| quorum sensing | switched off |
| further properties | no protease secretion |

Switched-Off Gene (According to P. aeruginosa Genome Project)

| | |
|---|---|
| gene number | not present in PAO genome |

Further Studies

As with the mutant having an insertion in Seq. ID 1, here too the PstI-digested genomic DNA of various P. aeruginosa isolates was hybridized with the sequence flanking the transposon insertion in a Southern hybridization.

The unknown DNA sequence is present not only in P. aeruginosa TB but also in most P. aeruginosa isolates studied. As expected, P. aeruginosa PAO gave no signal. A sequence alignment with the aid of BLASTN revealed a sequence approx. 80% identical to Seq. ID 3.

Summary

As with Seq. ID 1, this unknown sequence too is no clone-specific DNA of P. aeruginosa TB, since it also occurs in other isolates. A transposon insertion in this region which does not exist in P. aeruginosa PAO completely switched off P. aeruginosa TB quorum sensing. This is another indication of quorum sensing of P. aeruginosa TB, and possibly also of other P. aeruginosa isolates, being regulated by additional factors which are unknown from the PAO studied.

Seg. ID 3

Switched-Off Gene (According to P. aeruginosa Genome Project)

| | |
|---|---|
| gene number | not present in PAO genome |

Summary

Seq. ID 3 is 80% identical to Seq. ID 2 at the nucleotide level but represents, in contrast to the latter, the sequence of the complete gene. This is an example of a gene family which is present in more than 60% of all P. aeruginosa isolates and in many other Gram-negative bacteria but which is absent in the sequenced PAO strain. The similarity among the members of this gene family is approx. 80% at the nucleotide level and usually markedly higher still at the protein level.

Summary of the Results

Intracellular Survivability in Granulocytes

The inventors have studied more than 1000 transposon mutants for their intracellular survivability in granulocytes. Those P. aeruginosa mutants having the most distinct phenotypic deviations were collected and examined again. The most conspicuous bacteria whose phenotypic deviation was confirmed in this way were differentiated and quantified in more detail as to which proportion of the low survival rates measured can be attributed to low survivability in granulocytes and which proportion of the effect measured is due to a sensitivity to blood serum. Furthermore, the identified transposon mutants were studied for their invasivity in epithelial cells. Here, intracellular survivability in granulocytes and bacterial invasivity in epithelial cells were shown to be phenotypic properties unconnected with one another.

Sequencing of the flanking DNA regions of the genomically inserted transposons showed that genes which also occur in the P. aeruginosa PAO genome had been switched off in most of the P. aeruginosa TB transposon mutants which had not survived the selection experiment.

In transposon mutants whose sensitivity to blood serum was altered the cause seems to be a modification in the composition of the extracellular matrix or of the structure of the outer cell membrane. The causes of reduced survivability in granulocytes can first be attributed to defects in the defenses against oxidative stress or in the repair of oxidative damage to the DNA. Secondly, genes of the flagellar export system were found whose switching-off firstly disrupted flagella construction, but secondly also greatly reduced the intracellular survivability in granulocytes and invasivity in epithelial cells. The reason for this is a dual function of this export system which, in addition to its function in organizing flagella constructs, is likewise required for secretion of pathogenicity factors which enable P. aeruginosa to survive intracellularly.

Quorum Sensing

Quorum sensing functioning was studied by incubating the P. aeruginosa transposon mutants with an E. coli detector strain. The latter harbored a plasmid on which a luciferase was encoded downstream of a promoter to which an RNA polymerase was able to bind only in the presence of homoserine lactones. To this end, each individual mutant was studied separately in microtiter plates. The transposon mutants thus identified which were distinctly defective in production of long- and short-chain aliphatic homoserine lactones (AHL) were studied for their protease activity on two newly developed selection media. As was to be expected, there was a clear connection between AHL production and the secretion of proteases.

The genes found code for a multiplicity of various proteins the majority of which, however, are regulators usually having a previously unknown function. A second group of genes is associated with fatty acid metabolism. The results indicate that these gene products are essential for synthesizing the aliphatic side chains of homoserine lactones. Furthermore, a TonB-dependent iron receptor was found whose inactivation resulted in a complete stop of quorum sensing. Previously, quorum sensing was known only to regulate iron uptake, with the reverse mechanism not having been described previously. Furthermore, a protein was identified which is structurally similar to a β-lactamase and whose inactivation increases the amount of free homoserine lactones in the stationary growth phase and thus has the function of a homoserine lactonase. In addition, transposon insertions were found in two further DNA sequences not known from the sequencing of P. aeruginosa PAO which are, however, no clone-specific DNA of the TB strain but were also found in other P. aeruginosa isolates. With one exception, all genes found had previously not been assigned to quorum sensing. This indicates that the previously existing model for regulation of AHL production is very incomplete. The absence of two DNA sequences in P. aeruginosa PAO, whose inactivation resulted in a defective quorum sensing of the transposon mutant studied, was a surprising result and indicates that AHL production of different P. aeruginosa strains may be regulated differently. PAO lacks DNA sequences which occur in several other P. aeruginosa strains and which have there an essential function in regulation and production of homoserine lactones. The information obtained from studying P. aeruginosa PAO can thus describe quorum sensing in other P. aeruginosa strains only incompletely.

Experimental Part

Generation of Transposon Mutants

Bacterial Strains

Escherichia coli

| | |
|---|---|
| DH5: | F⁻, φ80, m80lacZΔM1S, Δ(lacYZA-argF)_{U169}, recA1, |
| | endA1, hsdR17 (r_{K}⁻; m_{K}⁺), supE44, λ, thi, gyrA, |
| | relA1 |
| | Use: storage of pTnMod-OGm and donor strain in |
| | triparental conjugation |
| HB101: | F⁻, leuB6, proA2, recA13, thi-1, ara-14, lacY1, |
| | galK2, xyl-5, mtI-1, rpsL20, supE44, hsdS20 (r_{B}⁻; |
| | m_{B}⁻) |
| | Use: as carrier of plasmid pRK2013; helper |
| | strain in triparental conjugation |
| Pseudomonas | serotype: 4 |
| aeruginosa TB: | pyocine type: 1h |
| | phage lysotyping: F8, M4, |
| | PS2, PS24, PS31, 352, 46b/2, |
| | 1214, Col21, F7, F10, PS21, |
| | PS73 |
| | A plasmid was not detected. |

Vectors
pTnMod-OGm

The vector pTnMod-OGm was first described by DENNIS & ZYLSTRA (1998). It is a “plasposon” having a mini Tn5 (containing a gentamicin resistance) and an oriTRP4 for conjugative transfer. The transposase remains on the plasmid during transposition. The transposed sequence in pTNMod-OGm additionally also comprises the origin of replication of the plasmid, meaning that, after transposition, the remaining plasmid can no longer be replicated and is thus lost. The origin of replication used, oriR pMB1, moreover is stably expressable in most E. coli strains but not in P. aeruginosa. Another advantage of this vector is its enabling the flanking regions of the inserted mini transposon to be mobilized again as a plasmid (plasmid rescue).

In this plasmid, an optimized signal sequence (V₄₀; V=A,G,C) was ligated into the KpnI cleavage site of pTnMod-OGm so as to flank the origin of replication. The plasmid is thus suitable for generating mutants having specific signal sequences.

pRK2013

pRK2013 is a “helper plasmid”. It codes for any genes required for a conjugation (tra and mob). Other plasmids having RP4 oriT can be mobilized with the aid of said helper plasmid, even if the donor strain itself does not have the genes required for conjugation (FIGURSKI D & HELINSKI D 1979).

Cultivation of Bacteria

The most frequently used culture medium for bacterial cultivation was Luria-Bertani (LB) medium. The bacteria usually cultivated from single colonies in 5 ml of LB (37° C., 250 rpm, 12-16 h). LB agar having an appropriate supplement of antibiotics was used as storable culture or for selection.

Selection media:

| | |
|---|---|
| E. coli DH5α pTnMod-OGm | LB + 25 μg/ml gentamicin |
| E. coli HB101 pRK2013 | LB + 50 μg/ml kanamycin |
| P. aeruginosa | LB + 50 μg/ml gentamicin |
| | and M9(glycerol) + 50 μg/ml |
| | gentamicin |

Triparental Conjugation

In this method a mobilizable plasmid is transferred from a donor strain which itself is not capable of conjugation to an acceptor strain with the aid of a helper strain which has the genes required for conjugation. It is important here only that the acceptor has no functioning restriction system.

| | |
|---|---|
| Donor strain | E. coli DH5α |
| Vector | pTnMod-OGm |
| Helper strain | E. coli HB101 with |
| | pRK2013 |
| Acceptor strains | P. aeruginosa TB |

P. aeruginosa TB was incubated on blood agar at 42° C. for 5-7 days, being transferred by inoculation every day. On the day before conjugation, the donor strain E. coli DH5α (with pTnMod-OGm) and the helper strain E. coli HB101 (with pRK2013) were streaked out on LB agar containing the appropriate antibiotic supplement.

The bacteria were resuspended in 10 mM MgSO₄ and adjusted to a density of 1.0 OD. Suspensions of donor, helper and acceptor were mixed in a 10:10:1 ratio and plated out on an LB agar plate. After incubation for several hours at 37° C., the bacteria were resuspended and aliquots were plated out on M9 (glycerol) agar plates supplemented with gentamicin. After approx. 2 days of incubation (37° C.), positive transposon mutants were transferred to a new M9 (glycerol) agar plate containing gentamicin. After incubation at 37° C. for 16 hours, the plates were stored at 4° C. for approx. 1 month, before introducing the P. aeruginosa mutants to further experiments or freezing them at −80° C. This storage was necessary in order to remove cotransferred E. coli which, although almost unable to grow using glycerol as carbon source, can nevertheless survive from the P. aeruginosa mutants. Said E. coli were not capable of surviving a starvation period of this length.

Preparation of Genomic DNA from Gram-Negative Bacteria

Genomic DNA was prepared from P. aeruginosa according to a method specially developed for Gram-negative bacteria (CHEN & KUO 1993). The DNA worked up in this way was used as template in the PCR or for preparing Southern blots.

Investigation of P. aeruginosa Transposon Mutants

Phagocytosis Assay

The P. aeruginosa TB transposon mutants generated were assayed for their survivability inside granulocytes. To this end, granulocytes were freshly prepared from blood and incubated with P. aeruginosa transposon mutants. The granulocytes were separated from extracellular bacteria by washing and filtering and then lysed so that the fraction of intracellular, viable P. aeruginosa became accessible to further analyses.

10⁷ freshly prepared granulocytes were incubated per selection mixture with 2·10⁸ cfu P. aeruginosa transposon mutants in RPMI1640 (with 10% human AB serum) at 37° C. for 2 hours.

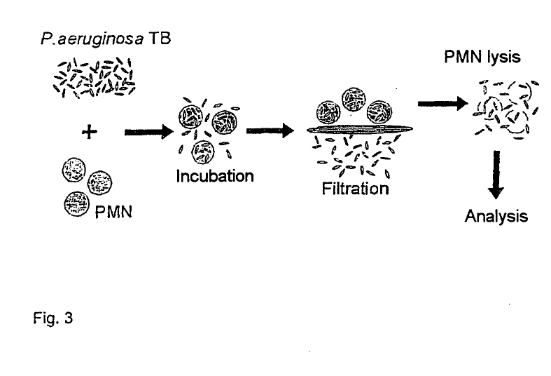

FIG. 3 depicts in a diagrammatic form the selection method for determining intracellular survivability in granulocytes of P. aeruginosa transposon mutants.

In a control mixture started in parallel, the bacteria were grown in RPMI 1640 without external selection to 2˜10⁸ bacteria. After the incubation had finished, the extracellular bacteria were removed from the intracellular ones by washing, centrifuging and filtering several times. The granulocytes were lysed and after incubation on complete medium for several hours, genomic DNA was prepared from the surviving bacteria. Accordingly, DNA was prepared from an aliquot of the control mixture.

Investigation of Quorum Sensing

The principle of the investigation method is to incubate the P. aeruginosa mutants together with an E. coli detector strain which has an episomally encoded luciferase expressed only in the presence of aliphatic homoserine lactones.

For this purpose, the transposon mutants were inoculated into microtiter plates and incubated at 37° C. The detector strain harboring the sensor plasmid was grown in LB+tetracycline to a density of OD 0.3-0.4 and an equal volume thereof was added to each P. aeruginosa transposon mutant. After incubation (37° C.) for 4 h, the luciferase activity was measured using a photon camera. The conspicuous mutants found in this investigation were then checked again in a second experiment on LB agar⁽¹⁾ together with the cross-streaked detector strain.

Determination of the Invasivity of P. aeruginosa Mutants into Epithelial Cells

Cell Culture of Eukaryotic Epithelial Cells (Chang Cells)

The medium (RPMI1640 containing 5% FCS) was removed from the starting culture (50 ml, 37° C., 5% CO₂) containing preconfluently grown cells which were then incubated with trypsin solution at 37° C. for 5-10 min. As soon as the cells had detached from the surface, RPMI1640 containing 5% FCS was added and the number of cells was determined in a Neubauer counting chamber, using a microscope. Aliquots were removed from this cell suspension for the experiments below.

Microscopic Study of Invasivity

The quickest possible way of determining the invasivity of different P. aeruginosa mutants is by microscopically studying adherent epithelial cells which had been incubated with the particular P. aeruginosa. To improve visibility, the cells were fixed with formaldehyde, with after incubation with bacteria (cells-to-bacteria ratio=1:100) for 1 hour, and stained with crystal violet. The counting was carried out under a microscope with a magnification of 1000×.

Quantification of P. aeruginosa Invasivity

The first steps of quantitatively determining the invasivity of P. aeruginosa strains are similar to those in the chapter above. The treatment of bacteria and epithelial cells corresponds to the abovementioned protocol. In this experiment too, the epithelial cells were incubated with a 100-fold excess of bacteria for a period to be determined experimentally.

After incubation (37° C., 5% CO₂), the epithelial cells were washed three times with RPMI1640 to remove the bacteria in the supernatant and incubated with RPMI1640 +100 μg/ml polymyxin B (37° C., 5% CO₂) in order to destroy adherent extracellular bacteria. The cells were then washed again twice with RPMI1640 and incubated with saponin solution (50 mg/ml) for 5-10 min to release the intracellular bacteria. Aliquots of this lysate and dilutions thereof were streaked out on LB agar, incubated at 37° C. overnight and the number of viable germs was determined.

Adherence of the bacteria to epithelial cells was determined by carrying out a comparable experiment in which, however, incubation with polymyxin B was dispensed with. The epithelial cells were washed with RPMI1640 only four times, before they were lysed.

Detection of P. aeruginosa Flagellin

When studying the intracellular survivability, a connection with the regulation of flagella construction was found for some transposon mutants. For a more detailed characterization, the following studies were carried out.

Immunological Study of Flagellar Mutants

The bacteria to be studied were grown in 5 ml of LB medium overnight and in each case 3 ml of bacteria suspension was removed for further studies. After the number of cells had been determined, 4·10¹⁰ bacteria were removed by centrifugation and resuspended in 1 ml of PBS. Aliquots of 10⁸ and 10⁷ bacteria (of a 1:10 dilution) were applied to a nitrocellulose membrane (Protran BA85, 0.45 μm) and dried at 37° C.

Flagellin was detected according to the protocol of the company Tropix, which can be used in general for immunological detections. The primary antibody used was a rabbit antibody against type b flagellin (Montie T, Univ. Tennessee, Knoxyille).

Staining of Flagella for Electron-Microscopic Study

P. aeruginosa from a culture incubated at 37° C. overnight were destroyed by adding formaldehyde (final concentration 1%) and fixed on a mica matrix. Staining was carried out by exposing the bacteria to 5 mM uranyl acetate (pH 7.0) for 5 min. After washing twice with distilled water, the preparation was dried and fixed to a copper support (the electron-microscopic study was carried out at GBF by Dr. M. Rohde).

Selection of STM Mutants

The P. aeruginosa transposon mutants were inoculated separately into microtiter plates from frozen glycerol cultures and incubated at 37° C. overnight. In each case 48 transposon mutants having different signal sequences were combined and studied for their survivability in granulocytes in a phagocytosis assay, with each group of 48 mutants being subjected in each case to two independent experiments.

After incubation at 37° C. overnight, the bacteria (selected and unselected samples) were resuspended in 10 mM MgSO₄ and exposed to the selection conditions for a second time. (The controls were suspended accordingly only in RPMI1640). The bacteria streaked out on LB agar were incubated at 37° C. overnight. Only after this second selection was genomic DNA of the surviving bacteria isolated.

The signal sequences of the transposons were then amplified from said DNA solutions by using PCR. The signal sequences thus obtained were restriction-digested with HindIII and the specific 40 bp sequences were purified via gel filtration (Sephadex G-100). The purified 40 bp sequences were labeled with DIG-ddUTP at the 3′ end with the aid of a terminal transferase.

The signal sequences of the donor plasmids were amplified with the aid of PCR and fixed in the form of dot blots to membranes. The 3′-endlabeled signal sequences from the phagocytosis assay were then hybridized to these prepared blots at 65° C. (16 h). The frequency of the individual signal sequences was determined by means of a light reaction. The X-ray films obtained were scanned and the blackening (od/mm²) of the individual dots was determined. The results were analyzed in MS Excel.

Plasmid Rescue

Plasmid rescue (AUSUBEL ET AL. 1987-95) is a method for transferring the flanking DNA of an inserted plasposon into episomally stable plasmids. For this purpose, genomic DNA of a transposon mutant was digested by a restriction endonuclease and the resulting fragments were converted to ring structures in a self-ligation reaction. Only the genomic sequence into which said plasposon had integrated had an origin of replication and an antibiotic cassette and was able to be stably expressed, after transformation into E. coli, on an appropriate selection medium. Said plasmids were then used for sequencing the flanking genomic DNA segments of the inserted transposon.

The genomic P. aeruginosa DNA was digested with PstI or Bc/I/BamHI and DNA was purified via a phenol/chloroform extraction with subsequent ethanol precipitation. The reaction was carried out by incubating the digested genomic DNA with T4 ligase. The ligation mixtures were concentrated in a vacuum concentrator and transformed into E. coli. The plasmids obtained in this way were sequenced to determine the DNA sequence flanking the transposon insertion.

### REFERENCES

Current Protocols in Molecular Biology (AUSUBEL ET AL, 1987-95)
BOLDUC, G. R., BOUCHET, V., JIANG, R. Z., GEISSELSODER, J., TRUONG-BOLDUC, Q. C., RICE, P. A., PELTON, S. I., GOLDSTEIN, R. (2000) Variability of outer membrane protein P1 and ist evaluation as a vaccine candidate against experimental otitis media due to nontypeable Haemophilus influenzae: an unambiguous, multifaceted approach. Infect Immun 68: 4505-4517
BOTZENHARDT K. & DÖRING G (1993): Ecology and epidemiology of Pseudomonas aeruginosa; 1-18. In: CAMPA M, BENDINELLI M, FRIEDMAN H (ed.): Pseudomonas aeruginosa as an opportunistic pathogen; Plenum Press New York
BRAVENY I, KRUMP-SCHMIDT W (1985): Pseudomonas aeruginosa; W. Zuckschwerdt Verlag, München
CHEN, W., Kuo, T. T. (1993) A simple and rapid method for the preparation of gram negative bacterial genomic DNA Nuc Ac Res 21:2260.
Chiang, S. L., Mekalanos, J. J., Holden, D. W. (1999) In vivo genetic analysis of bacterial virulence. Annu Rev Microbiol 53, 129-154.
COSTERTON J,W., CHENG, K. J., GEESEY, G. G., LADD, T. I., NICKEL, J. C., DASGUPTA, M., MARRIE, T. J. (1987) Bacterial biofilms in nature and disease: Ann. Rev. Microbiol. 41: 435-464.
DEKIEVIT, T. R., IGLEWSKI, B. H. (2000) Bacterial Quorum Sensing in Pathogenic Relationships. Infect Immun 68: 4839-4849.
DENNIS, J. J., ZYLSTRA, G. J. (1998) Plasposons: modular self-cloning minitransposon derivatives for rapid genetic analysis of gram-negative bacterial genomes. Appl Environ Microbiol 64:2710-15
DIRUSSO, C. C., BLAcK, P. N. (1999) Long-chain fatty acid transport in bacteria and yeast. Paradigms for defining the mechanism underlying this protein-mediated process. Mol Cell Biochem 192:41-52
FAREWELL, A., DIEZ, A. A., DIRUSSO, C. C., NYSTROM, T. 1996 Role of the Escherichia coli FadR regulator in stasis survival and growth phase-dependent expression of uspA, fad and fab genes. J Bacteriol 178:6443-6450.
FIGURSKI, D., HELINSKI, D. (1979). Replication of an origin-containing derivative of plasmid RK2 dependent on a plasmid function provided in trans. Proc Natl Acad Sci USA 1979 76:1648-1652.
HASSETT, D. J., MA, J. F., ELKINS, J. G., MC DERMOTT, T. R., OCHSNER, U. A., WEST, S. E., HUANG, C. T., FREDERICKS, J., BURNETT, S., STEWART, P. S., MCFETERS, G., PASSADOR, L., IGLEWSKI, B. H. (1999) Quorum sensing in Pseudomonas aeruginosa controls expression of caltalase and superoxide dismutase genes and mediates biofilm susceptibility to hydrogen peroxide. Mol Microbiol 34:1082-1093.
Hensel, M., Shea, J. E., Gleeson, C., Jones, M. D., Dalton, E., Holden, D. W. (1995) Simultaneous identification of bacterial virulence genes by negative selection. Science 269: 400-403.
Hensel, M. (1998) Whole genome scan for habitat-specific genes by signature-tagged mutagenesis. Electrophoresis 19, 608-612.
HOBBS, M., MATTICK, J. S. (1993): Common components in the assembly of type 4 fimbriae, DNA transfer systems, filamentous phage and protein-secretion apparatus: a general system for the formation of surface-associated protein complexes. Mol Microbiol 10: 233-243
HØIBY, N., DÖRING, G., SCHIØTZ, P. O. (1986) The role of immune complexes in the pathogenesis of bacterial infections Ann Rev Microbiol 40: 29-53.
HORAN T C (1986): Nosocomial infection surveillance 1984, Morbid. Mortal. Wkl. Rep.; 35:17-29.
LUMPPIO, H. L., SHENVI, N. V., SUMMERS, A. O., VOORDOUW, G., KURTZ, D. M. (2001) Rubrerythrin and rubredoxin oxidoreductase in Desulfovibrio vulgans: novel oxidative stress protection system. J Bacteriol 183: 101-108.
MACNAB, R. M. (1992): Genetics and Biogenesis of bacterial flagella. Annu Rev Genet 26:131-158.
MAHAN, J. M., SLAUCH, J. M., MEKALANOS, J. J. (1993) Selection of bacterial virulence genes that are specifically induced in host tissues Science 259: 686-688
MAHAN, J. M., TOBIAS, J. W., SLAUCH, J. M., HANNA, P. C., COLLIER, R. J., MEKALANOS, J. J. (1995) Antibiotic-based selection for bacterial genes that are speciffically induced during infection of a host Genetics 92: 669-673.
MARTIN, P. R., HOBBS, M., FREE, P. D., JESKE, Y., MATTICK, J. S. (1993): Characterization of pilQ, a new gene required for the biogenesis of type 4 fimbriae in Pseudomonas aeruginosa. Mol Microbiol 9: 857-868
Mecsas, J. (2002) Use of signature-tagged mutagenesis in pathogenesis studies. Curr Opin Microbiol 5, 33-37
MINAMINO, T., MACNAB, R. M. (1999) Components of the Salmonella flagellar export apparatus and classification of export substrates. J Bacteriol 181:1388-1394.
OHBA A, MIZuSHIMA T, KATAYAMA T, SEKIMIZU K (1997): Amounts of proteins altered by mutations in the dnaA gene of Escherichia coli. FEBS Lett 404:125-128.
PIANZZOLA, M. J., SOUBES, M., TOUATI, D. (1996) Overproduction of the rbo gene product from Desulfovibrio species suppresses all deleterious effects of tack of superoxide dismutase in Escherichia coli. J Bacteriol 178: 6736-6742
PIER G. D. (1985) Pulmonary disease associated with Pseudomonas aeruginosa in cystic fibrosis: current status of the host—bacterium interaction J Infect Dis 151: 575-580.
POLLAK, M. (1985) Pseudomonas aeruginosa; 1236-1250. In: MANDELL, G. L., DOUGLAS, R. G. JR., BENNET, J. E. (ed.) Principles and practice of infectious diseases, 2. Auflage; John Wiley & Sons, New York
RAINEY, P. B., HEITHOFF, D. M., MAHAN, M. J. (1997) Single-step conjugative cloning of bacterial gene fusions involved in microbe-host interactions Mol Gen Genet 256:84-87.
SLAUCH, J. M., MAHAN, M. J., MEKALANOS, J. J. (1994) in vivo expression technology for selection of bacterial genes specifically induced in host tissues Methods Enzymol 235:481-492.
STOREY, D. G., UJACK, E. E., RABIN, H. R., MITCHELL, I. (1998): Pseudomonas aeruginosa lasR transcription correlates with the transcription of lasA, lasB, and toxA in chronic lung infections associated with cystic fibrosis. Infect Immun 66:2521-8.
TAKASE, H., NITANAI, H., HOSHINO, K., OTANI, T. (2000): Requirement of the Pseudomonas aeruginosa tonB gene for high-affinity iron acquisition and infection. Infect Immun 68: 4498-4504.
WEI, Y., LEE, J. M., RICHMOND, C., BLATTNER, F. R., RAFALSKI, J. A., LAROSSA, R. A. (2001) High-density microarray-mediated gene expression profiling of Escherichia coli. J Bacteriol 183: 545-556.
YOUNG, G. M., SCHMIEL, D. H., MILLER, V. L. (1999) A new pathway for the secretion of virulence factors by bacteria: the flagellar export apparatus functions as a protein-secretion system. Proc Natl Acad Sci USA 96:6456-6461.

1. An isolated or recombinant nucleic acid, characterized by any of the sequences according to Seq. ID 1, Seq. ID 2 or Seq. ID 3 or a degenerated or modified sequence homologous thereto with corresponding function, said modifications comprising deletions, insertions and/or substitutions of amino acids, in particular point mutations.

2. A protein encoded by a sequence as claimed in claim 1 or an essentially functionally equivalent protein or peptide homologous thereto, in particular one associated with sequence modification due to deletion, insertion or substitution of single and/or multiple amino acids, sequence-extending additions of single and/or multiple amino acids and/or chemical derivatization, in particular of the terminal amino acids.

3. The use of the nucleic acids or nucleotide sequences PA0740, PA1104, PA1288, PA1322, PA1441, PA1452, PA1572, PA1992, PA2591, PA3344, PA4621, PA5040, PA5349 and/or PA5415 of the microorganism Pseudomonas aeruginosa, denoted according to the nomenclature of the Pseudomonas aeruginosa Community Annotation Project, and/or of the nucleic acids or nucleic acid sequences as claimed in claim 1, or of fragments or modified sequences of said nucleic acids or nucleic acid sequences with corresponding function or of the in each case corresponding proteins endogenous to Pseudomonas aeruginosa, all of which are essential for the viability of said microorganism in humans or animals, as targets for the development of diagnostic agents for identifying the virulence of a Pseudomonas aeruginosa strain.

4. The use of the nucleic acids or nucleotide sequences PA0740, PA1104, PA1288, PA1322, PA1441, PA1452, PA1572, PA1992, PA2591, PA3344, PA4621, PA5040, PA5349 and/or PA5415 of the microorganism Pseudomonas aeruginosa, denoted according to the nomenclature of the Pseudomonas aeruginosa Community Annotation Project, and/or of the nucleic acids or nucleic acid sequences as claimed in claim 1, or of fragments or modified sequences of said nucleic acids or nucleic acid sequences with corresponding function or of the in each case corresponding proteins endogenous to Pseudomonas aeruginosa, all of which are essential for the survivability of said microorganism in humans or animals, as targets for the development of therapeutics for Pseudomonas aeruginosa strains.

5. The use of the nucleic acids or nucleotide sequences PA0740, PA1104, PA1288, PA1322, PA1441, PA1452, PA1572, PA1992, PA2591, PA3344, PA4621, PA5040, PA5349 and/or PA5415 of the microorganism Pseudomonas aeruginosa, denoted according to the nomenclature of the Pseudomonas aeruginosa Community Annotation Project, and/or of the nucleic acids or nucleic acid sequences as claimed in claim 1, or of fragments or modified sequences of said nucleic acids or nucleic acid sequences with corresponding function or of the in each case corresponding proteins endogenous to Pseudomonas aeruginosa, all of which are essential for the survivability of said microorganism in humans or animals, as targets for the development of vaccines for Pseudomonas aeruginosa strains.

6. The use as claimed in claim 3, characterized in that the sequences PA1104 and/or PA1452 are used instead of or in connection with PA1441.

7. The use as claimed in claim 3, characterized in that, instead of the proteins, proteins homologous thereto or protein fragments of said proteins and homologous variants are used, in particular those associated with sequence modification due to deletion, insertion or substitution of single and/or multiple amino acids, sequence-extending additions of single and/or multiple amino acids and/or chemical derivatization, in particular of the terminal amino acids.

8. An antibody directed against by any of the nucleic acid sequences PA0740, PA1104, PA1288, PA1322, PA1441, PA1452, PA1572, PA1992, PA2591, PA3344, PA4621, PA5040, PA5349 and PA5415 of the microorganism Pseudomonas aeruginosa, denoted according to the nomenclature of the Pseudomonas aeruginosa Community Annotation Project, or against fragments or modified sequences of said nucleic acids or nucleic acid sequences with corresponding function, or against a protein encoded by any of the nucleic acid sequences as claimed in claim 1 or of the sequences above or against at least one functional fragment of such a protein, in particular for the use in determining the virulence of Pseudomonas aeruginosa strains.

9. A vaccine, comprising at least one protein encoded by any of the nucleic acid sequences PA0740, PA1104, PA1288, PA1322, PA1441, PA1452, PA1572, PA1992, PA2591, PA3344, PA4621, PA5040, PA5349 and PA5415 of the microorganism Pseudomonas aeruginosa, denoted according to the nomenclature of the Pseudomonas aeruginosa Community Annotation Project, or of the nucleic acid sequences as claimed in claim 1, or at least one functional fragment of any said proteins, or at least one fusion protein based on said protein or functional part of said protein.

10. A vaccine, comprising at least one of the nucleic acids according to sequences PA0740, PA1104, PA1288, PA1322, PA1441, PA1452, PA1572, PA1992, PA2591, PA3344, PA4621, PA5040, PA5349 and PA5415 of the microorganism Pseudomonas aeruginosa, denoted according to the nomenclature of the Pseudomonas aeruginosa Community Annotation Project, or of fragments or modified sequences of the nucleic acids or nucleic acid sequences above with corresponding function, or of the nucleic acids as claimed in claim 1, in a modified modification readable in mammalian cells and in relation to a promoter readable in mammalian cells.

11. The vaccine as claimed in claim 10, characterized in that the at least one gene together with the corresponding promoter has been ligated into a plasmid.

12. The vaccine as claimed in claim 10, additionally comprising customary additives and excipients and preferably at least one adjuvant.

13. The use as claimed in claim 4, characterized in that the sequences PA1104 and/or PA1452 are used instead of or in connection with PA1441.

14. The use as claimed in claim 4, characterized in that, instead of the proteins, proteins homologous thereto or protein fragments of said proteins and homologous variants are used, in particular those associated with sequence modification due to deletion, insertion or substitution of single and/or multiple amino acids, sequence-extending additions of single and/or multiple amino acids and/or chemical derivatization, in particular of the terminal amino acids.

15. The use as claimed in claim 5, characterized in that the sequences PA1104 and/or PA1452 are used instead of or in connection with PA1441.

16. The use as claimed in claim 5, characterized in that, instead of the proteins, proteins homologous thereto or protein fragments of said proteins and homologous variants are used, in particular those associated with sequence modification due to deletion, insertion or substitution of single and/or multiple amino acids, sequence-extending additions of single and/or multiple amino acids and/or chemical derivatization, in particular of the terminal amino acids.

17. The vaccine as claimed in claim 9, characterized in that the at least one gene together with the corresponding promoter has been ligated into a plasmid.

18. The vaccine as claimed in claim 9, additionally comprising customary additives and excipients and preferably at least one adjuvant.
